(19) United States
(12) Patent Application Publication
(10) Pub. No.: US 2004/0170981 A1
(43) Pub. Date: Sep. 2, 2004

(54) Real-time polymerase chain reaction using large target amplicons

(76) Inventors: Keith McKenney, Potomac, MD (US); Lidja Gillmeister, Fairfax, VA (US); Kristina Marlowe, Fairfax, VA (US); David Armistead, Arlington, VA (US)

Correspondence Address: FLESHNER & KIM, LLP, P.O. BOX 221200, CHANTILLY, VA, 20153, US

(21) Appl. No.: 10/361,004

(22) Filed: Feb. 10, 2003

(51) Int. Cl. C12Q 1/68; C12P 19/34

(52) U.S. Cl. 435/6; 435/91.2

(57) ABSTRACT

The present invention relates to methods for analyzing a target nucleic acid sequence in a biological material. More particularly, the present invention relates to methods for analyzing a target nucleic acid sequence by real time polymerase chain reaction using nucleic acid primers that are separated by at least about 750 nucleic acid residues in the target sequence.

### BACKGROUND OF THE INVENTION

1. Field of the Invention

The present invention relates to methods for analyzing a target nucleic acid sequence in a biological material. More particularly, the present invention relates to methods for analyzing a target nucleic acid sequence by real time polymerase chain reaction using nucleic acid primers that are separated by at least about 750 nucleic acid residues in the target sequence.

2. Background of the Related Art

PCR (polymerase chain reaction) is a method for increasing the concentration of a segment of a target sequence in a mixture of nucleic acid sequences without cloning or purification. (See K. B. Mullis et al., U.S. Pat. Nos. 4,683,195 and 4,683,202).

This process for amplifying the target sequence consists of introducing two oligonucleotide primers to the sample containing the desired target nucleic acid sequence, followed by thermal cycling in the presence of a DNA polymerase. The two primers are complementary to their respective strands of the target sequence. To effect amplification, the genetic material within the sample is first denatured and then the primers are annealed to their complementary sequences within the target molecule. Following annealing, the primers are extended with a polymerase so as to form a new pair of complementary strands.

The steps of denaturation, annealing and extension can be repeated many times (i.e., denaturation, annealing and extension constitute one “cycle”; there can b numerous “cycles”) to obtain a high concentration of an amplified segment of the desired target sequence. The length of the amplified segment of the desired target sequence is determined by the relative positions of the primers with respect to each other, and therefore, this length is a controllable parameter. Because the desired amplified segments of the target sequence become the predominant sequences (in terms of concentration) in the mixture, they are said to be “PCR amplified”.

With PCR, it is possible to amplify a single copy of a specific target sequence in genomic DNA to a level detectable by several different methodologies (e.g., hybridization with a labelled probe; incorporation of biotinylated primers followed by avidin-enzyme conjugate detection; incorporation of ³²P-labelled deoxynucleotide triphosphates, e.g., dCTP or dATP, into the amplified segment). In addition to genomic DNA, any oligonucleotide sequence can be amplified with the appropriate set of primer molecules.

End-point PCR is a polynucleotide amplification protocol. The amplification factor that is observed is related to the number (n) of cycles that have occurred and the efficiency of replication at each cycle (E), which, in turn, is a function of the priming and extension efficiencies during each cycle. Amplification has been observed to follow the form Eⁿ, until high concentrations of the PCR product have been made.

At these high product concentrations, the efficiency of replication tends to drop significantly. It has been suggested that this is probably due to the displacement of the primers by the longer complementary strands of the PCR product. At concentrations in excess of 10⁻⁸ M, the rate of the two complementary PCR amplified product strands finding each other during the priming reactions becomes sufficiently fast that it may occur before or concomitantly with the extension step of the PCR process. This ultimately leads to a reduced priming efficiency, and, consequently, a reduced cycle efficiency. Continued cycles of PCR lead to declining increases of PCR product molecules, until the PCR product eventually reaches a plateau concentration (the “end-point”), usually a concentration of approximately 10⁻⁸ M. As a typical reaction volume is about 100 microliters, this corresponds to a yield of about 6×10¹¹ double stranded product molecules.

Real-time PCR is also a polynucleotide amplification protocol, but PCR product analysis occurs simultaneously with amplification of the target sequence. Detecting agents, such as DNA dyes or fluorescent probes, can be added to the PCR mixture before amplification and used to analyze PCR products during amplification. Sample analysis occurs concurrently with amplification in the same tube within the same instrument. This combined approach decreases sample handling, saves time, and greatly reduces the risk of product contamination, as there is no need to remove the samples from their closed containers for further analysis. The concept of combining amplification with product analysis has become known as “real-time” or “quantitative” PCR. (See, e.g., WO/9746707A2, WO/9746712A2 and WO/9746714A1).

Originally, monitoring fluorescence each cycle of PCR involved the use of ethidium bromide. See Higuchi et al., “Simultaneous amplification and detection of specific DNA sequences,” Bio/Technology 10:413-417 (1992); Higuchi et al., “Kinetic PCR analysis: real time monitoring of DNA amplification reactions,” Bio/Technology 11:1026-1030 (1993). In that system, fluorescence was measured once per cycle as a relative measure of product concentration. Ethidium bromide detects double stranded DNA; thus, if the desired target nucleic acid sequence is present, fluorescence intensity increases with temperature cycling (otherwise no fluorescence). Furthermore, the cycle number where an increase in fluorescence is first detected increases inversely proportionally to the log of the initial target sequence concentration. Other fluorescent systems have since been developed that are capable of providing additional data concerning the nucleic acid concentration.

A significant limitation in the use of real-time PCR is the length of the target nucleic acid sequence. That is, as the target amplicon length increase, the efficiency of real-time PCR decreases. Practical limits for target amplicon length in most commercially available PCR systems are generally less than 500 bp, usually in the range of 80-200 bp. Larger amplicons have been obtained by some, but to date there remains a need for routinely amplifying large target sequences in real time PCR.

Each of the above references is incorporated by reference herein where appropriate for teachings of additional or alternative details, features and/or technical background.

### SUMMARY OF THE INVENTION

An object of the invention is to solve at least the problems and/or disadvantages of the relevant art, and to provide at least the advantages described hereinafter.

Accordingly, it is an object of the present invention to provide methods for analyzing a target nucleic acid sequence by real time polymerase chain reaction using nucleic acid primers that are separated by at least about 750 nucleic acid residues in the target sequence. Other objects, features and advantages of the present invention will be set forth in the detailed description of preferred embodiments that follows, and in part will be apparent from the description or may be learned by practice of the invention. These objects and advantages of the invention will be realized and attained by the compositions and methods particularly pointed out in the written description and claims hereof.

In accordance with these and other objects, a first embodiment of the present invention is directed to a method for analyzing a target nucleic acid sequence, comprising: (i) adding to a biological material an effective amount of at least two nucleic acid primers that hybridize under stringent conditions to predetermined sequences of the target sequence and are separated by at least about 750 nucleic acid residues, (ii) amplifying the target nucleic acid sequence by a polymerase chain reaction which comprises adding a polymerase to the biological material and primers to form an amplification mixture and thermally cycling the amplification mixture between at least one denaturation temperature and at least one elongation temperature, and (iii) detecting and quantifying said target nucleic acid sequence. According to this embodiment of the present invention, during the thermal cycling, the elongation temperature is not more than about 70° C. and the denaturation temperature is not more than about 95° C., and the amplification mixture is maintained at the denaturation temperature for a period of not more than about 30 seconds and at the elongation temperature for a period of not less than about 1 minute.

Additional advantages, objects, and features of the invention will be set forth in part in the description which follows and in part will become apparent to those having ordinary skill in the art upon examination of the following or may be learned from practice of the invention. The objects and advantages of the invention may be realized and attained as particularly pointed out in the appended claims.

### BRIEF DESCRIPTION OF THE DRAWINGS

The invention will be described in detail with reference to the following drawings in which like reference numerals refer to like elements wherein:

FIG. 1 shows forward and reverse primers useful in preparing large target amplicons based on the genomic nucleic acid sequence of human Parvovirus B19.

FIG. 2 shows forward and reverse primers useful in preparing large target amplicons based on the genomic nucleic acid sequence of hepatitis B virus.

FIG. 3 shows forward and reverse primers useful in preparing large target amplicons based on the genomic nucleic acid sequence of porcine parvovirus.

FIG. 4 shows forward and reverse primers useful in preparing large target amplicons based on the genomic nucleic acid sequence of Sinbis virus.

FIG. 5 shows forward and reverse primers useful in preparing large target amplicons based on the genomic nucleic acid sequence of West Nile virys.

FIGS. 6A and 6B show forward and reverse primers useful in preparing large target amplicons based on the genomic nucleic acid sequence of the 16S ribosomal RNA gene and the 23S ribosomal RNA gene of Escherichia coli.

FIGS. 7A and 7B show forward and reverse primers useful in preparing large target amplicons based on the genomic nucleic acid sequence of the 18S ribosomal RNA gene and the 25S ribosomal RNA gene of yeast (S. cerevisiae).

FIG. 8 shows forward and reverse primers useful in preparing large target amplicons based on the nucleic acid sequence of human mitochondrial DNA.

### DETAILED DESCRIPTION OF PREFERRED EMBODIMENTS

A. Definitions

Unless defined otherwise, all technical and scientific terms used herein are intended to have the same meaning as is commonly understood by one of ordinary skill in the relevant art.

As used herein, the singular forms “a,” “an,” and “the” include the plural reference unless the context clearly dictates otherwise.

As used herein, the term “biological material” is intended to mean any substance derived or obtained from a living organism. Illustrative examples of biological materials include, but are not limited to, the following: cells; tissues; blood or blood components; proteins, including recombinant and transgenic proteins, and proetinaceous materials; enzymes, including digestive enzymes, such as trypsin, chymotrypsin, alpha-galactosidase and iduronodate-2-sulfatase; immunoglobulins, including mono and polyimmunoglobulins; botanicals; food and the like. Preferred examples of biological materials include, but are not limited to, the following: ligaments; tendons; nerves; bone, including demineralized bone matrix, grafts, joints, femurs, femoral heads, etc.; teeth; skin grafts; bone marrow, including bone marrow cell suspensions, whole or processed; heart valves; cartilage; corneas; arteries and veins; organs, including organs for transplantation, such as hearts, livers, lungs, kidneys, intestines, pancreas, limbs and digits; lipids; carbohydrates; collagen, including native, afibrillar, atelomeric, soluble and insoluble, recombinant and transgenic, both native sequence and modified; chitin and its derivatives, including NO-carboxy chitosan (NOCC); stem cells, islet of Langerhans cells and other cells for transplantation, including genetically altered cells; red blood cells; white blood cells, including monocytes; and platelets. Additional examples of biological materials include forensic samples, human or animal remains, stomach contents, mummified remains of a once-living organism, fossilized remains, a product of manufacture containing or previously in contact with a biological material, and fomites.

B. Particularly Preferred Embodiments

A first particular preferred embodiment of the present invention is directed to a method for analyzing a target nucleic acid sequence in a biological material, comprising:

(i) adding to a biological material an effective amount of at least two nucleic acid primers, wherein these nucleic acid primers hybridize under stringent conditions to two predetermined nucleic acid sequences of the target nucleic acid sequence that are separated by at least about 750 nucleic acid residues,

(ii) amplifying the target nucleic acid sequence by a polymerase chain reaction which comprises adding a polymerase to the biological material and primers to form an amplification mixture and then thermally cycling the amplification mixture between at least one denaturation temperature and at least one elongation temperature; and

(iii) detecting and quantifying said target nucleic acid sequence.

According to this preferred embodiment of the present invention, the elongation temperature is not more than about 70□ C and the denaturation temperature is not more than about 95□ C. Additionally, according to this preferred embodiment of the present invention, during each thermal cycle, the amplification mixture is maintained at the denaturation temperature for a period of not more than about 30 seconds and at the elongation temperature for a period of not less than about 1 minute.

According to preferred embodiments of the present invention, the target nucleic acid sequence preferably contains between about 500 and about 50,000 nucleic acid residues. More preferably, the target nucleic acid sequence contains between about 1000 and about 10,000 nucleic acid residues, even more preferably between about 2000 and about 5000 nucleic acid residues and most preferably between about 2500 and about 5000 nucleic acid residues.

The nucleic acid primers are each selected based on their ability to generate the desired target nucleic acid sequence under the appropriate PCR conditions. Accordingly, each primer must be specific for the desired target nucleic acid sequence. Similarly, each primer must be selected so that they are not self-complementary or complementary to another primer (or probe, if present).

According to preferred embodiments of the present invention, the sequences on the target sequence that correspond to the two primer sequences are separated by at least 750 nucleic acid residues. Preferably, the sequences which correspond to the primers are separated by at least about 1000 nucleic acid residues, more preferably at least about 2000 nucleic acid residues, even more preferably at least about 3000 nucleic acid residues, still even more preferably at least about 4000 nucleic acid residues and most preferably at least about 5000 nucleic acid residues. According to an alternative embodiment of the present invention, the sequences on the target sequence that correspond to the two primer sequences are separated by at only about 500 nucleic acid residues.

The polymerase chain reaction employed in the inventive methods is performed according to the methods and techniques known to those skilled in the art, i.e., a nucleic acid primer pair is added to the biological material containing the sequence of interest to form an amplification mixture that is then thermally cycled for a sufficient period of time to amplify the desired sequence. The thermal cycling generally comprises cycling the amplification mixture between at least one denaturation temperature and at least one elongation temperature. Preferably, the thermal cycling comprises cycling the amplification mixture between at least one denaturation temperature, at least one annealing temperature and at least one elongation temperature.

Specific temperatures for use in denaturation, elongation and/or annealing may be determined empirically by one skilled in the art based, for example, on the specific target sequence being amplified and the particular probes employed. Likewise, the specific time(s) that the amplification mixture is maintained at the various denaturation, elongation and/or annealing temperature(s) may be determined empirically by one skilled in the art based on similar considerations.

According to particularly preferred embodiments of the present invention, the elongation temperature selected for use in the PCR of the inventive methods is not more than about 70° C. More preferably, the elongation temperature selected is between about 60° C. and about 69° C., and even more preferably between about 65° C. and about 69° C. Most preferably, the elongation temperature employed in the PCR of the inventive methods is about 68° C.

According to additional preferred embodiments of the present invention, the denaturation temperature selected for use in the PCR of the inventive methods is not more than about 95° C. More preferably, the denaturation temperature selected is between about 90° C. and about 95° C., and even more preferably between about 93° C. and about 95° C. Most preferably, the denaturation temperature employed in the PCR of the inventive methods is about 94° C.

According to other preferred embodiments of the present invention, when the thermal cycling includes an annealing temperature, the annealing temperature selected is about 5-10° C. below the melting temperature of the primers being employed. Preferably, the annealing temperature selected is not more than about 65° C. More preferably, the annealing temperature selected is between about 57° C. and about 63° C., and even more preferably between about 58° C. and about 62° C. Most preferably, the annealing temperature employed in the PCR of the inventive methods is about 60° C.

According to additional preferred embodiments of the present invention, during each thermal cycle, the amplification mixture is maintained at the elongation temperature for a period of not less than about 1 minute. More preferably, during each thermal cycle, the amplification mixture is maintained at the elongation temperature for a period of not less than about 2 minutes, and even more preferably for a period of not less than about 3 minutes.

According to particularly preferred embodiments of the present invention, the amplification mixture is maintained at the elongation temperature for a period of not less than about 2 minutes during the first cycle of the thermal cycling, and then the period during which said amplification mixture is maintained at the elongation temperature is increased by a period of about 5 seconds for each successive thermal cycle. Thus, for example, according to such embodiments of the present invention, if the amplification mixture was maintained at the elongation temperature for a period of 2 minutes during the first cycle of the thermal cycling, it would be maintained at the elongation temperature for a period of 2 minutes, 5 seconds for the second cycle, 2 minutes, 10 seconds for the third cycle, 2 minutes, 15 seconds for the fourth cycle, and so on until the thermal cycling is completed.

According to additional preferred embodiments of the present invention, during each thermal cycle, the amplification mixture is maintained at the denaturation temperature for a period of not more than about 1 minute. More preferably, during each thermal cycle, the amplification mixture is maintained at the denaturation temperature for a period of not more than about 45 seconds, and even more preferably for a period of not more than about 30 seconds, and still even more preferably for a period of not more than about 20 seconds. Most preferably, during each thermal cycle, the amplification mixture is maintained at the denaturation temperature for a period of not more than about 15 seconds, such as a period of about 10 seconds.

According to still other preferred embodiments of the present invention, when the thermal cycling includes an annealing temperature, the amplification mixture is maintained at the annealing temperature for a period of not less than about 30 seconds. More preferably, according to such embodiments, during each thermal cycle, the amplification mixture is maintained at the annealing temperature for a period between 30 seconds and 2 minutes, and even more preferably for a period of not less than about 45 seconds. Most preferably, during each thermal cycle, the amplification mixture is maintained at the annealing temperature for a period of about 1 minute.

The number of thermal cycles employed in the PCR of the inventive methods may be determined empirically by one skilled in the art depending, for example, on the suspected concentration of the target sequence of interest in the biological material being tested. According to preferred embodiments of the present invention, the amplification mixture is subjected to at least about 30 cycles of thermal cycling, and even more preferably at least about 40 cycles. Most preferably, the amplification mixture is subjected to at least about 50 cycles of thermal cycling.

The polymerase employed in the PCR of the inventive methods may be any of the suitable polymerases known to those skilled in the art. Preferably, the polymerase employed is a thermostable polymerase, i.e. a polymerase that is not adversely affected by the higher temperatures involved in thermal cycling. More preferably, the polymerase may be a Taq polymerase, or a suitable derivative thereof and/or a proof-reading polymerase.

According to particularly preferred embodiments of the present invention, at least two polymerases are employed in the PCR of the inventive methods. Preferably, at least one of the polymerases is a Taq polymerase or a suitable derivative thereof, such as TaqMan DNA polymerase (available from Applied BioSystems), and the other polymerase is a proof-reading polymerase, such as ProofStart DNA polymerase (available from Qiagen).

According to certain preferred embodiments of the present invention, the amplification mixture further contains at least one thermostable inorganic pyrophosphatase. Suitable amounts of thermostable inorganic pyrophosphatase may be determined empirically by one skilled in art. Generally, when present, the ratio of thermostable inorganic pyrophosphatase to Taq polymerase is at least about 1:20, more preferably at least about 1:10 and even more preferably at least about 1:5.

The remaining parameters employed in the PCR of the inventive methods, such as the primer concentration (generally about 100-500 nM and preferably about 200 nM)), magnesium concentration (generally 1.5-6 mM and preferably about 1.5 mM of magnesium sulfate and/or magnesium chloride), deoxyribonucleotide triphosphates (dNTP) concentration (generally about 0.2-0.4 mM each and preferably about 0.2 mM each), probe concentration (if present, generally about 50-800 nM, and preferably about 100 nM), may each be determined empirically by one skilled in the art using any of the known methods and techniques.

According to certain particularly preferred embodiments of the present invention, the deoxyribonucleotide triphosphates (dNTP) that are employed in the PCR of the inventive methods are selected from the group consisting of C, T, G and A. Preferably, substantially no dUTP is present in the amplification mixture of the inventive methods. According to still further preferred embodiments, substantially no uracil N-glycosylase is present in the amplification mixture of the inventive methods.

According to certain particularly preferred embodiments of the present invention, the amplification mixture futher comprises at least one buffer solution. Suitable buffer solutions are known and available to those skilled in the art. Particularly preferred buffer solutions include pH modifying buffers, such as buffers containing Tris-HCl, and buffers which maintain salt concentration, particular magnesium concentration, such as buffers containing KCl and/or (NH₄)₂SO₄.

After amplification using PCR, the first and second target nucleic acid sequences are detected and quantified. This detecting and quantifying may be conducted using any of the methods and techniques known to those skilled in the art. For example, detecting and quantifying of the first and second nucleic acid sequences may be conducted by adding a suitable detecting agent, such as an intercalating dye, directly to the amplification mixture or by adding a suitable nucleic acid probe to the mixture, preferably either a suitable nucleic acid probe in combination with a detecting agent or a suitable nucleic acid probe having a detectable label covalently or ionically attached thereto or complexed therewith.

Preferably, the target nucleic acid sequence is detected by adding at least one nucleic acid probe to the biological material being tested. Any nucleic acid probe employed in the inventive methods should contain sufficient nucleic acid residues to hybridizes selectively under stringent conditions to a specific desired nucleic acid sequence, i.e. suitable probes will generally contain at least 16 nucleic acid residues, and preferably hybridizes selectively under stringent conditions to a specific nucleic acid sequence of the target nucleic acid sequence that is not the same as the nucleic acid sequence of any of the primers. Suitable nucleic acid probes include, but are not limited to, 5′ nuclease probes, hairpin probes, adjacent probes, sunrise probes and scorpion probes.

### EXAMPLES

The following examples are illustrative, but not limiting, of the present invention. Other suitable modifications and adaptations are of the variety normally encountered by those skilled in the art and are fully within the spirit and scope of the present Invention.

### Example 1

Purpose: To demonstrate linear amplification of B19 DNA.

Materials: 1. B19 virus, titer 7.6×10¹¹ iu/ml from Bayer;

2. SNAP whole blood DNA isolation kit;

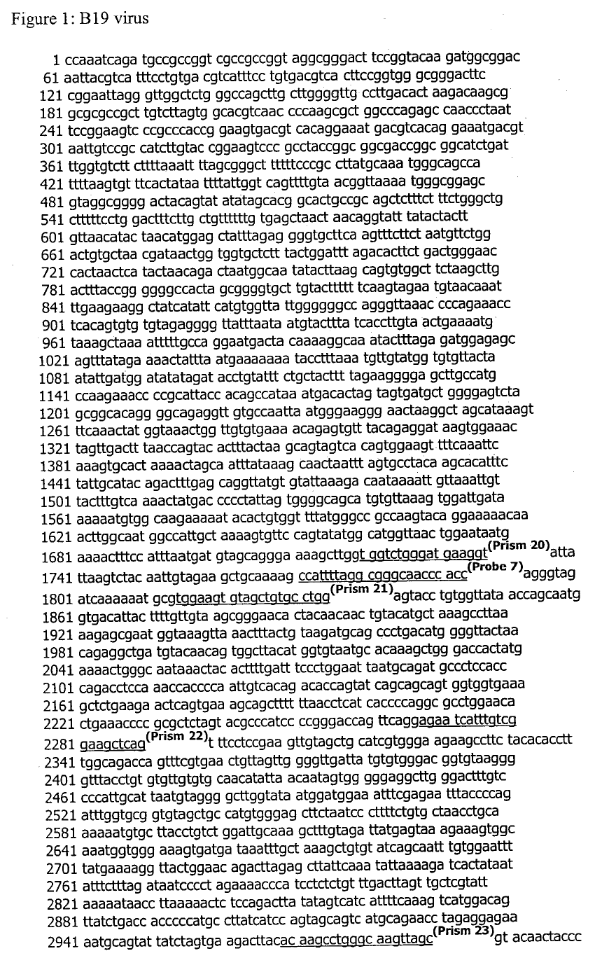

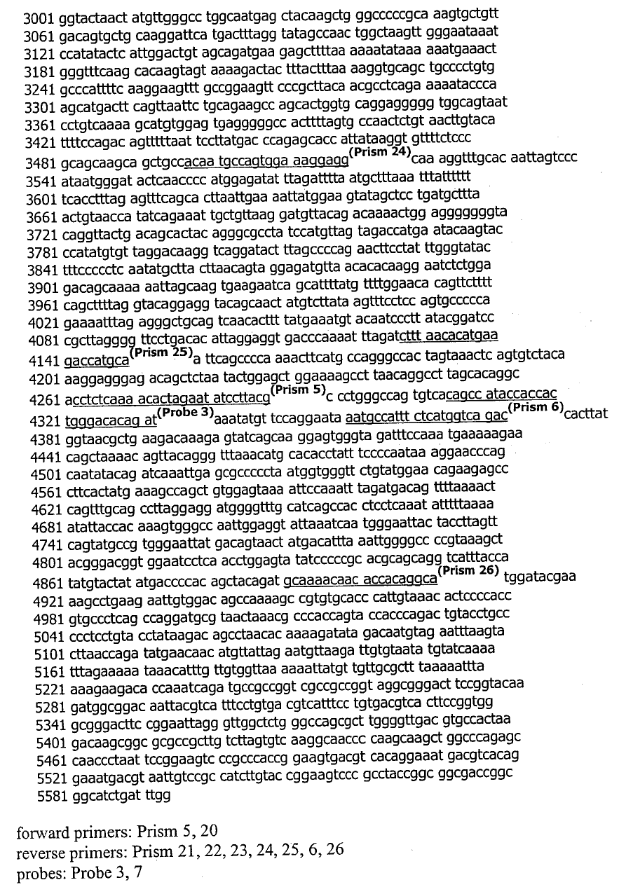

3. Forward Primer: Prism 5 (FIG. 1);

4. Reverse Primer: Prism 6 (FIG. 1);

5. Probe 3 (FIG. 1) labeled with FAM at 5′ end and TAMRA at 3′ end;

6. TaqMan Universal Master Mix, (ABI; cat. no. 4304437);

7. DNASE, RNASE free water;

8. ABI 96 well plate and adhesive cores;

9. ANI 7000.

Procedure: 1. Followed SNAP protocol for extraction of 100 μl B19 sample, eluted in 100 μl TE;

2. Diluted primers to 18 μM with TE;

3. Diluted probe to 5 μM with TE;

4. Prepared the following master mix:

TaqMan Master Mix: 25 μl;

Prism 5: 2.5 μl;

Prism 6: 2.5 μl;

Taqman Probe 2.5 μl;

Water: 12.54 μl;

5. Added 45 μl of master mix per well;

6. Serially diluted B19 DNA, adding water to the NTC well;

7. Sealed and centrifuged the plate at 2300 rpm for about 30 seconds;

8. Ran PCR program for 50 cycles.

Results: A standard dilution curve was observed for B19 infected plasma, validating primer pair Prism 5 and Prism 6 with Probe 3.

### Example 2

Purpose: To examine irradiated and unirradiated samples containing PPV using a 549 bp amplicon.

Materials: 1. PPV (irradiated at 0 kGy, 50 kGy, 65 kGy, 75 kGy or 85 kGy);

2. SNAP Protein Degrader;

3. Cell Lysis Buffer;

4. Tris-HCl;

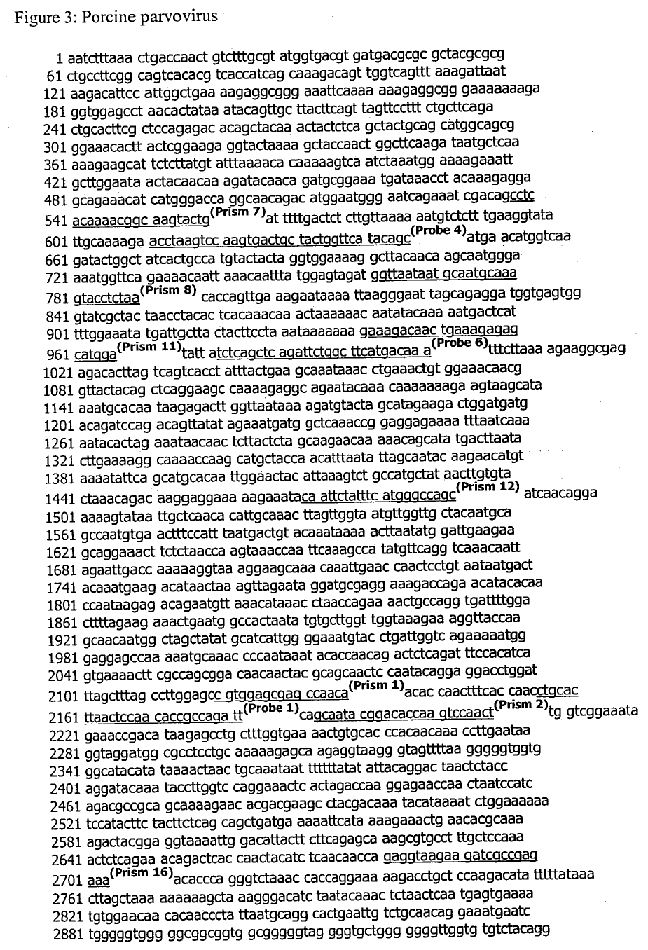

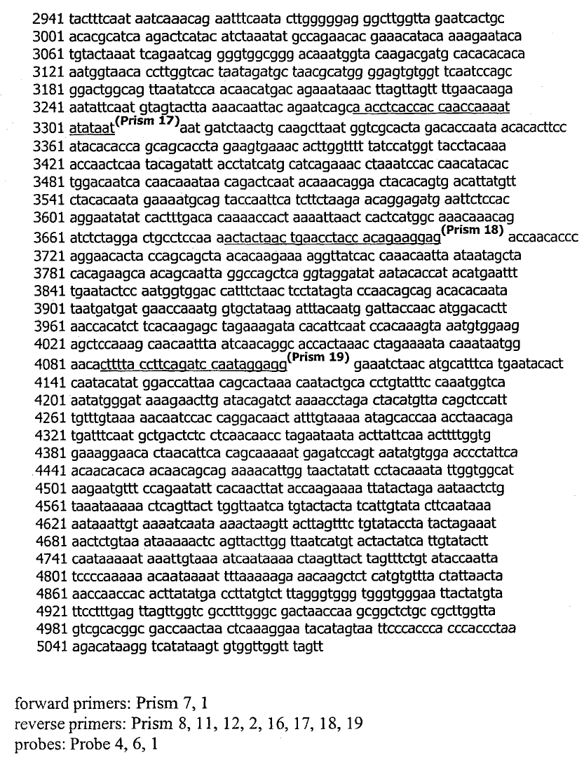

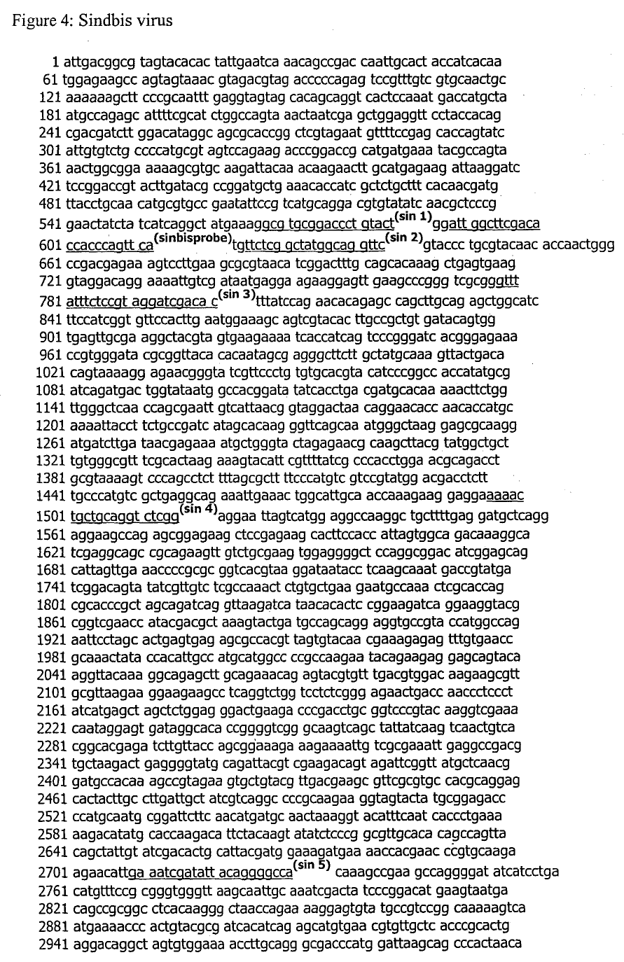

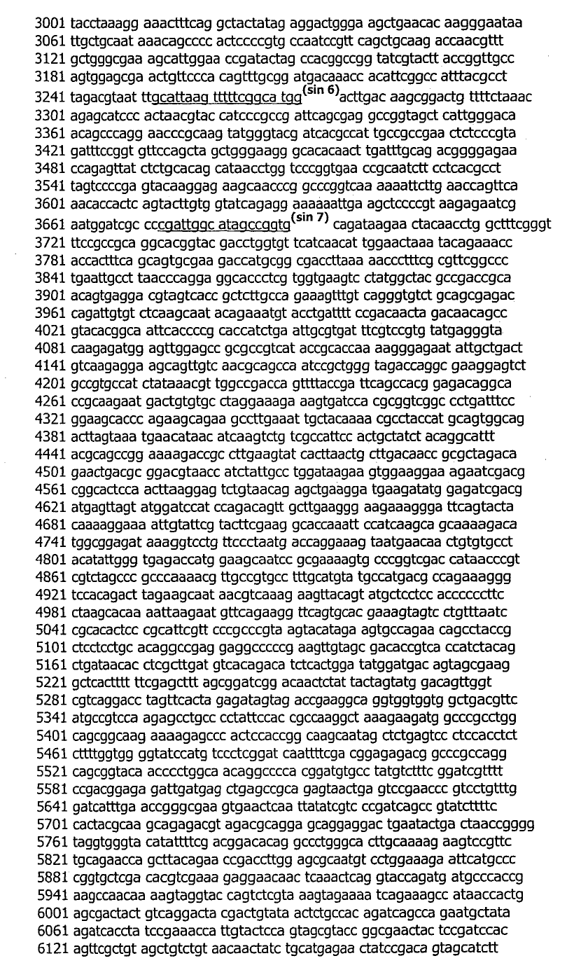

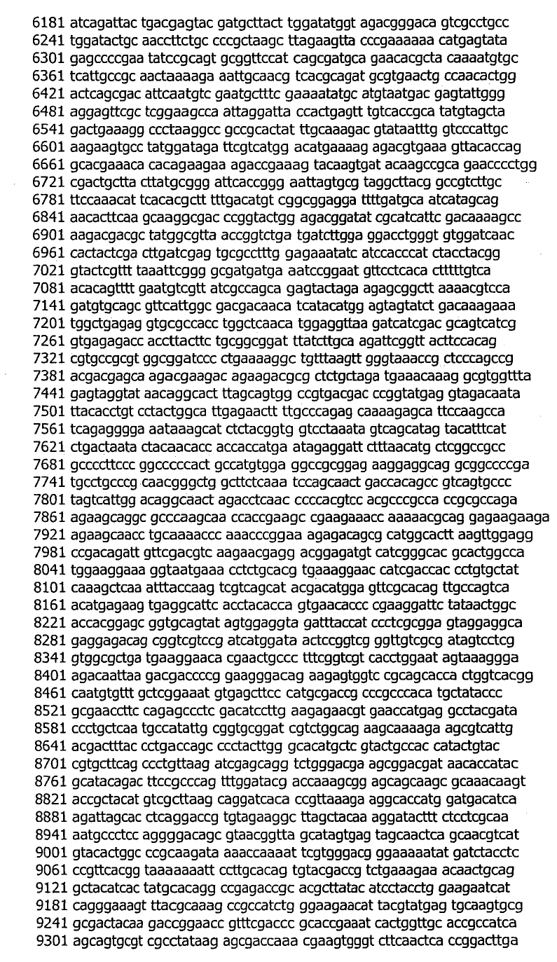

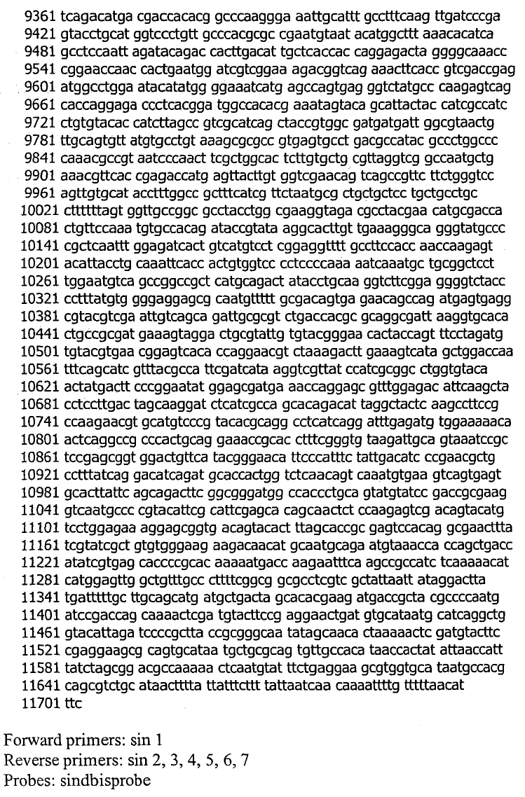

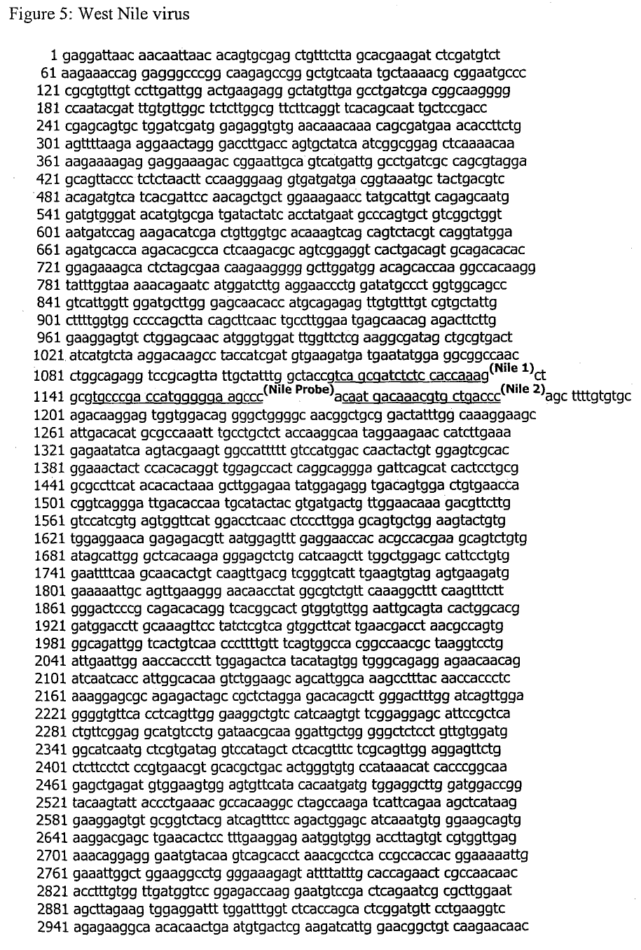

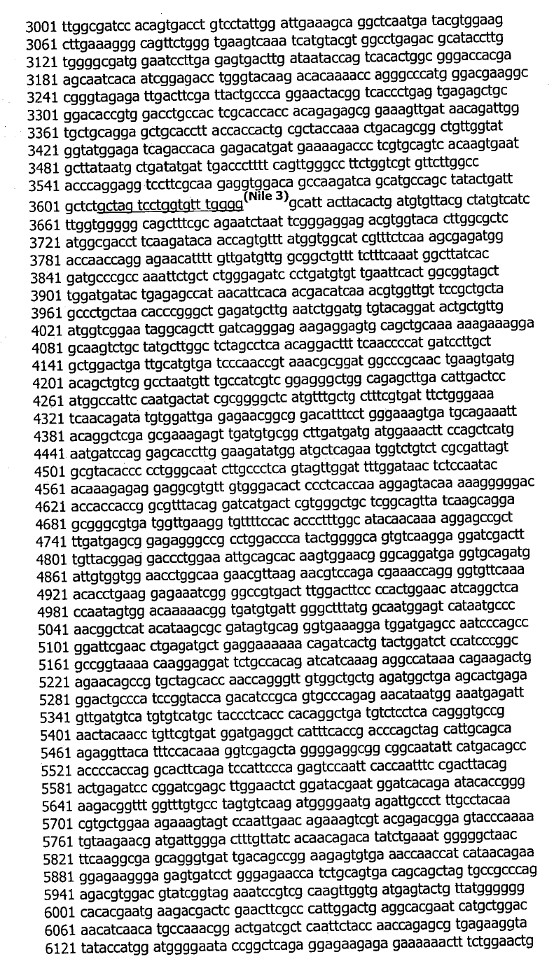

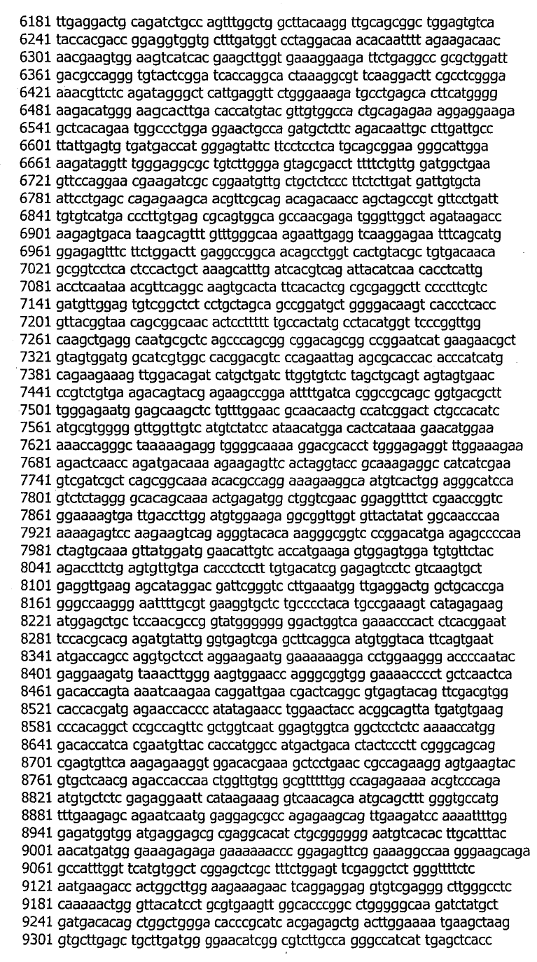

5. Primers: Prism 11 and Prism 12 (FIG. 3); and

6. Probe 6 (FIG. 3).

Procedure: 1. To 100 μl viral sample, added 50 μl tris-HCl buffer, 60 μl protein degrader, and 200 μl cell lysis buffer;

2. Mixed and incubated for 25 minutes (5 minutes at 70° C.);

3. Diluted samples to 1/50, 1/500, 1/5000, 1/25000, 1/50000, 1/250000 and 1/500000;

4. Ran PCR for 55 cycles.

Results: Results showed that unirradiated material had regular dilution series curves, irradiated material (50 kGy) behaved differently, dilute material did not amplify showing a reduction in the number of copies of the target sequence.

### Example 3

Purpose: To determine effects of gamma irradiation (0 kGy sample, 50 kGy sample, mixture of 0+50 kGy sample and 75 kGy sample) on samples containing PPV analyzed by PCR.

Materials: 1. PPV (irradiated at 0 kGy, 50 kGy or 75 kGy);

2. Primers: Prism 11 & Prism 12, Probe 6 FIG. 3);

3. Primers: Prism 1 & Prism 2, Probe 1 (FIG. 3).

Procedure: 1. Diluted samples containing PPV to 1/100, 1/1000, 1-2000, 1/10000, 1/20000, 1/40000 and 1/400000 (0 kGy, 50 kGy, 0+50 kGy and 75 kGy);

2. Ran PCR program for 55 cycles.

Results: Irradiaition to 50 kGy of PPV material reduced amplification of 549 bp amplicon.

### Example 4

| Purpose: | To examine the relative effectiveness | | |
|---|---|---|---|
| | of Qiagen and Taqman | | |
| | reagents on samples containing PPV. | | |
| Materials: | 1. PPV DNA (phenol extracted); | | |
| | 2. Taq PCR Core Kit; | | |
| | 3. ProofStart DNA polymerase; | | |
| | 4. Taqman Universal PCT Master Mix; | | |
| | 5. Prism 1, 2, 11 and 17 (FIG. 3); | | |
| | 6. Probes 1 and 6 (FIG. 3); | | |
| | 7. Agarose; | | |
| | 8. TAE; | | |
| | 9. EtBr. | | |
| Procedure: | 1. Prepared the following four master mixes: | | |
| | a. Qiagen: | 1 | 2 |
| | 10 × buffer: | 30 μl | 25 μl |
| | dNTP's: | 9 μl | 7.5 μl |
| | pA: | 8.34 μl | 6.95 μl |
| | pB: | 8.34 μl | 6.95 μl |
| | taq: | 6 μl | 5 μl |
| | H₂O: | 187.32 μl | 156.1 μl |
| | probe: | 15 μl | 12.5 μl |
| | b. Taqman: | 3 | 4 |
| | Master Mix: | 150 μl | 125 μl |
| | pA: | 15 μl | 12.5 μl |
| | pB: | 15 μl | 12.5 μl |
| | probe: | 15 μl | 12.5 μl |
| | H₂O: | 69 μl | 57.5 μl |

2. Pipetted 44 μl of master mix 1 into row D, wells 1 and 2; row E, wells 1 and 2; and row H, well 1, of a well plate;

3. Pipetted 44 μl of master mix 2 into tow D, wells 3 and 4; and row E, wells 3 and 4, of a well plate;

4. Pipetted 44 μl of master mix 3, into row F, wells 1 and 2; row G, wells 1 and 2; and row H, well 3, of a well plate;

5. Pipetted 44 μl of master mix 4 into tow F, wells 3 and 4; and row G, wells 3 and 4, of a well plate;

6. Added 1 μl of ProofStart taq to row D, wells 1-4 and row F, wells 1-4 and added 1 μl water to remaining wells;

7. Added 5 μl water to tow H, wells 1 and 3 and added 5 μl PPV DNA to remaining wells;

8. Ran PCR for 40 cycles.

Results: Qiaqen Master with ProofStart taq produced functional large amplicons in realtime PCR with PPV DNA more efficiently than the TaqMan master mix.

### Example 5

Purpose: To examine the effects of proofstart in amplifying large amplicons and to examine the effects of 50 kGy irradiation on PPV.

Materials: 1. PPV DNA (irradiated to 0 kGy and 50 kGy);

2. Taq PCR Core Kit;

3. Proofstart DNA polymerase;

4. Prism 11, 16 and 17 (FIG. 3);

5. Agarose;

6. Ethidium Bromide;

7. TAE buffet.

Procedure: 1. Set up PCR master mix as follows:

10× buffer: 50 μl

dNTP's: 15 μl

pA: 13.9 μl (primer 11)

taq: 10 μl

water: 347.2 μl

2. Placed aliquots into PCR tubes;

3. Added either primer 16 or 17 to PCR tubes;

4. Added PPV DNA (diluted to 1:100) to each PCR tube:

5. Added 10 μl proofstart to half of the samples (2 at 0 kGy and 2 at 50 kGy);

6. Performed PCR (about 55 cycles)

7. Poured a 1% gel and ran at 100 V for 20 minutes.

Results: Addition of a proofreading polymerase resulted in improved amplication of longer amplicons. Delay in amplification of target sequence in irradiated samples is proportional to damage done to viral genetic material.

### Example 6

Purpose: To examine the effect of TSP concentration on amplification of large target amplicons in gamma irradiated and unirradiated PPV.

Materials: 1. TSP (cat. no. M02965);

2. Qiagen Core kit;

3. ProofStart DNA polymerase;

4. PPV (irradiated to 0 kGy or 50 kGy).

Procedure: 1. Prepared a master mix (standard PCR set-up) for each (TSP: Taq 1:20, 1:10, 1:5);

2. Added 43.61 μl of each master mix (TSP titration) to PCR tubes;

3. Added 1.39 μl of primers 16, 17 or 19 (FIG. 3) to appropriate PCT tubes;

4. Added 5 μl water to the negative control, which contained primer pair 11, 16 (FIG. 3);

5. Diluted PPV 1:100;

6. Added PPV to PCR tubes;

7. Performed PCR;

8. Poured a 1% gel and ran at 100 V for 20 minutes.

Results: Under these conditions, addition of TSP resulted in increased amplification of target amplicons in both irradiated and unirradiated samples, but irradiation of PPV resulted in decreased amplification of target amplicons.

### Example 7

Purpose: To examine the effects of gamma irradiation on amplification of PPV target amplicons of various sizes.

Materials: 1. PPV DNA (irradiated to 0 kGy or 50 kGy);

2. Taq PCR Core Kit;

3. ProofStart DNA Polymerase;

4. Prism 11, 16, 17, 18 and 19 (FIG. 3);

5. Agarose;

6. TAE;

7. Ethidium Bromide.

Procedure: 1. Prepared PCR Master Mix as follows:

10× Buffer 5 μl

dNTPs 1.5 μl

pA 1.39 μl

pB 1.39 μl

taq 1 μl

water 33.72 μl

PPV 5 μl.

2. Alliquoted samples into PCR tubes;

3. Ran PCR;

4. Poured a 1% agarose gel and ran at 120 V for about 1.5 hours.

Results: Irradiation to 50 kGy resulted in decreased amplification of larger target amplicons.

### Example 8

Purpose: To examine PCR sensitivity and determine log reduction of PPV in samples irradiated to 50 kGy and having a starting concentration of 2.5×10⁷ gEq.

Materials: 1. Standard PCR reagents (Qiacen Core Kit, TSP, Proofstart, etc.);

2. Primers 11 and 17 (FIG. 3);

3. PPV extract.

Procedure: 1. Prepared master mix with primers 11 and 17;

2. Performed a 10 fold dilution series from 10⁷ to 10⁰ of PPV extract;

3. Pipetted 45 μl of master mix into PCR tubes;

4. Pipetted 5 μl of each PPV dilution into appropriated PCR tubes;

5. Added 5 μl water to control;

6. Ran PCR;

7. Ran samples in 1% agarose at 100V for about 47 minutes.

Results: Irradiation of sample to 50 kGy resulted in decreased amplification of target amplicon across all concentration ranges.

### Example 9

Purpose: To examine PCR sensitivity and determine log reduction of PPV irradiated to 50 kGy and having a starting concentration of 2.5×10⁷ gEq.

Materials: 1. TSP;

2. Standard PCR kit (Qiacen with ProofStart Polymerase);

3. Primers 11 and 19 (FIG. 3);

4. PPV Extract (Irradiated to 0 kGy and 50 kGy).

Procedure: 1. Prepared master mix with primers 11 and 19;

2. Performed a 10 fold dilution series from 107 to 10⁰ of PPV extract;

3. Pipetted 45 μl of master mix into PCR tubes;

4. Pipetted 5 μl of each PPV dilution into appropriated PCR tubes;

5. Added 5 μl water to control;

6. Ran PCR as follows: 95° C. for 2 minutes (1 cycles)

94° C. for 10 seconds (40 cycles)

60° C. for 1 minute (40 cycles)

68° C. for 2 minutes (40 cycles);

7. Cooled to 4° C.;

8. Ran samples on 1% agarose gel in 1×TAE and 5 μl/100 ml ethidium bromide at 100 V for 52 minutes (5 μl on gel).

Results: Irradiation to 50 kGy resulted in decreased amplification of target amplicon across all concentration ranges. For unirradiated samples, relative band strength of observed target amplicon decreased with decreasing concentration.

### Example 10

Purpose: Primer validation for B19 using probe 7 and various primers.

Materials: 1. B19 IGIV Paste (irradiated to 0 kGY or 50 kGy);

2. EXB;

3. Proteinase;

4. yeast tRNA

5. phenol chloroform isoamyl alcohol;

6. 3M NaAc;

7. isopropanol;

8. 70% EtOH;

9. TE buffer;

10. Prisms 5, 6, 20, 21, 22, 23, 24, 25, 26 (FIG. 1);

11. Qiagen reagents;

12. Ampligold Taq;

13. ProofStart Polymerase;

14. Agarose;

15. TAE;

16. Ethidium Bromide.

Procedure: 1. Prepared a Master Mix as follows:

Buffer 5 μl

DNTP 1.5 μl

Taq 1 μl

DNA 5 μl

water 34.72 μl

2. Pipetted Master Mix into PCR tubes;

3. Added the following primer pairs to appropriate PCR tubes: 20&21; 20&22; 20&23; 20&24; 20&25; 20&6; 20&26; 5&6;

4. Ran PCR;

5. ran 1% gel for about 1 hour.

Results: All tested primers yielded desired target amplicons.

### Example 11

Purpose: Use of PCR multiplexing with target amplicons of about 112 bp and about 2.4 kbp for B19 virus in samples irradiated to 0 kGy or 50 kGy.

Materials: 1. TSP thermostable inorganic pyrophosphatase

2. Standard PCR reagents;

3. B19 viral extract (irradiated to 0 kGy and 50 kGy);

4. Prisms 5, 6, 20 and 25 (FIG. 1);

5. Taq;

6. ProofStart Polymerase.

Procedure: 1. Prepared standard PCR set-up with 3× master mixes, for each primer set (primer sets: 5&6; 20&25; 5&6; and 20&25);

2. Prepared appropriate PCR tubes containing the following primer pairs: (5, 6) 0 kGy; (5, 6) 50 kGy; (20, 25) 0 kGy; (20, 25) 50 kGy; (5, 6) & (20, 25), 0 kGy; and (5, 6) and (20, 25), 50 kGy;

3. Added 5 μl B19 to PCR tubes containing 45 μl of appropriate master mix;

4. Added 5 μl water to control;

5. Ran PCR.

6. Ran samples on 1% aragose gel at 100 V for about 17 minutes.

Results: PCR multiplexing is effective for mixtures containing large target amplicons and small target amplicons. Irradiation to 50 kGy resulted in decreased amplification of the large target amplicon relative to the small target amplicon.

### Example 12

Purpose: Irradiated and unirradiated samples containing B119 viral material were examined using teal time PCR.

Materials: 1. B19 viral material (irradiated to 0 kGy and 50 kGy);

2. Prism pairs (20, 21) and (20, 26) (FIG. 1);

3. Qiagen PCR reagents;

4. Qiagen ProofStart;

5. Agarose;

6. TAE (1×);

7. sample loading buffer (SLB).

Procedure: 1. Prepared standard samples containing primer pairs with 10¹¹ to 10¹ dilution series;

2. Ran PCR (40 cycles);

3. Ran gel on 1% agarose (8 μl PCR product, 1 μl SLB) at 100 V for about 20 minutes.

Results: Irradiation to 50 kGy resulted in decreased amplification of large target amplicon. Unirradiated samples exhibited a regular dilution pattern.

### Example 13

Purpose: To investigate the effect of gamma irradiation on samples containing HBV and irradiated to 50 kGy.

Materials: 1. HBV (irradiated to 0 kGy and 50 kGy);

2. Taq PCR Core Kit;

3. ProofStart DNA polymerase;

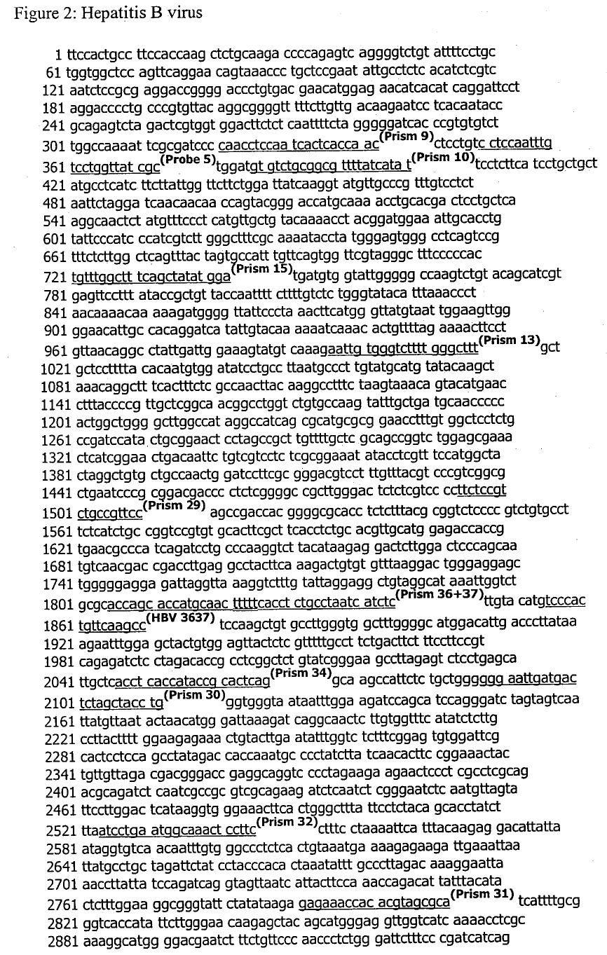

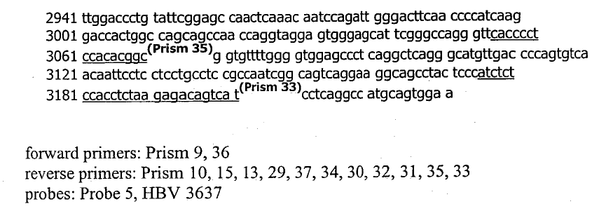

4. Prisms 3, 4, 9, 10, 15, 29, 30, 31, 36 and 37 (FIG. 2);

5. Agarose;

6. TAE Buffer;

7. ethidium bromide.

Procedure: 1. Prepared PCR master mix as follows:

10×PCR buffer 5 μl

dNTPs 1.39 μl

primers 1.39 μl

taq 1 μl

ProofStart 1 μl

water 33.22 μl

TSP 0.5 μl

2. Aliquoted 43.61 μl of master mix into PCR tubes. Appropriate tubes contained the following primer pairs: (3, 4); (9, 10); (9, 15); (9, 29); (9, 30); (9, 31); (36, 37); and (9, 31), for both irradiated and unirradiated samples;

3. Added 5 μl HBV per tube (irradiated or unirradiated);

4. Ran PCR as follows:

50° C. for 2 minutes (one cycle)

95° C. for 2 minutes (one cycle)

94° C. for 10 seconds (40 cycles)

60° C. for 1 minute (40 cycles)

68° C. for 2 minutes, five seconds (40 cycles);

5. Ran 1% agarose gel (9 μl sample+1 μl sample buffet) at 100v for about 20 minutes.

Results: Irradiated samples showed no amplification of large target amplicons, indicating degradation of HBV genetic material by irradiation to 50 kGy.

### Example 14

Purpose: To investigate the effect of gamma irradiation on samples containing HBV DNA and irradiated to 50 kGy.

Materials: 1. HBV DNA material (irradiated to 0 kGy and 50 kGy);

2. Taq PCR Core Kit (Qiagen, cat. no. 201223);

3. ProofStart Taq Polymerase (Qiagen, cat. no. 20);

4. Prisms 10, 13, 30, 36 and 37 (FIG. 2);

5. Agarose;

6. TAE Buffer;

7. Ethidium Bromide.

Procedure: 1. Prepared the following master mix:

10× buffer 60 μl

dNTP 18 μl

primer 36 16.68 μl

Taq 12 μl

ProofStart 12 μl

water 440.64 μl;

2. Pipetted 46.61 μl of master mix into PCR tubes;

3. Added 1.39 μl of reverse primer (10, 13, 30 or 37) and 2 μl HBV DNA (0 kGy and 50 kGy) to appropriate tubes;

4. Ran PCR for 50 cycles;

5. Poured a 1% agarose gel (8 μl PCR product+1 μl sample buffer) at 100 V for about 20 minutes.

Results: Irradiated samples showed no amplification of large target amplicons, indicating degradation of HBV DNA by irradiation to 50 kGy.

### Example 15

Purpose: HBV amplification of nested primer set (about 80 bp, 400 bp and 697 bp) in samples containing ascorbate, including digestion of 0 kGy and 50 kGy samples with exonuclease I prior to PCR amplication.

Materials: 1. HBV DNA (irradiated to 0 kGy and 50 kGy, with and without ascorbate);

2. Primer sets: (9, 10); (9, 14) and (9, 13) (FIG. 2);

3. Exonuclease I;

4. Standard PCR reagents.

Procedure: 1. Diluted HBV samples to 1/500, 1/2000 and 1/10000;

2. Digested 1 μl raw HBV extract in 0.25 μl Exonuclease I, 10 μl 10× Exonuclease I buffer and 88.75 μl water at 37° C. for 30 minutes, inactivated at 80° C. for 20 minutes;

3. Dilutes digested HBV to 1/2000 and 1/10000;

4. Ran 55 cycles PCR.

Results: Irradiated showed no amplification of large target amplicon (697 bp), indicating degradation of HBV DNA by irradiation to 50 kGy.

### Example 16

Purpose: To investigate the amount of bacterial and fungal DNA present in pulverized tendon samples.

Materials: 1. E. Coli samples (tendon)—0 or 50 kGy+stabilizer (6.65×1¹⁰ CFU/μl);

2. C. Albicans samples (tendon)—0 or 50 kGy+stabilizer (3.55×10⁹ CFU/μl);

3. Staph. Aureus samples;

4. Control tendon;

5. Dneasey tissue kit (Qiagen, cat. no. 69504);

6. Taq PCR Core Kit (Qiagen, cat. no. 201223);

7. ProofStart Taq Polymerase (Qiagen, cat. no. 202205);

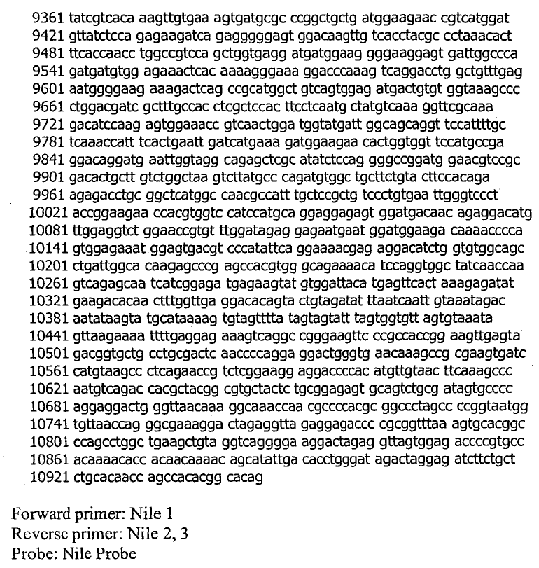

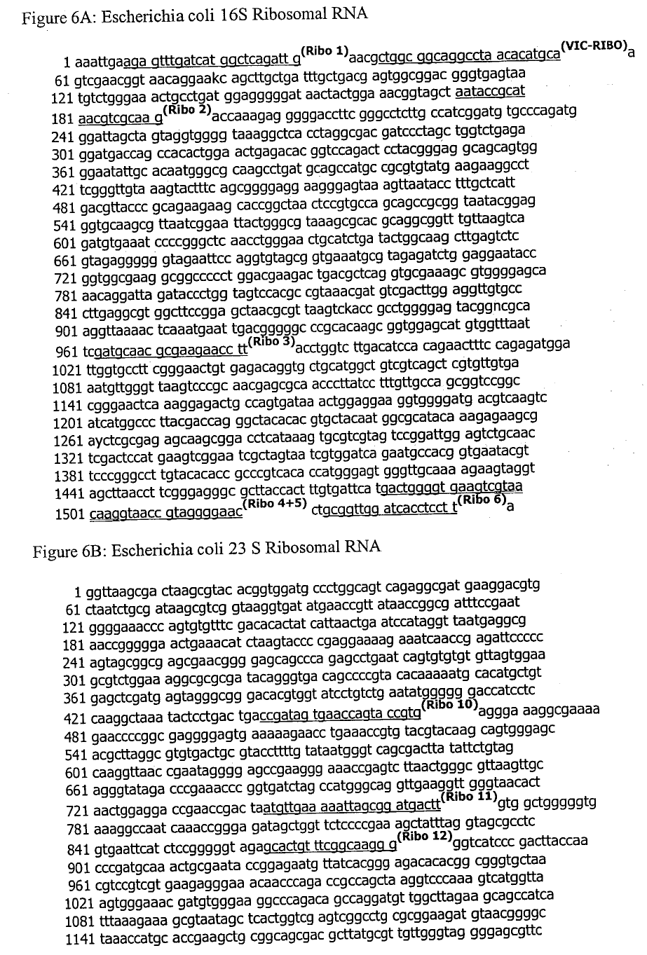

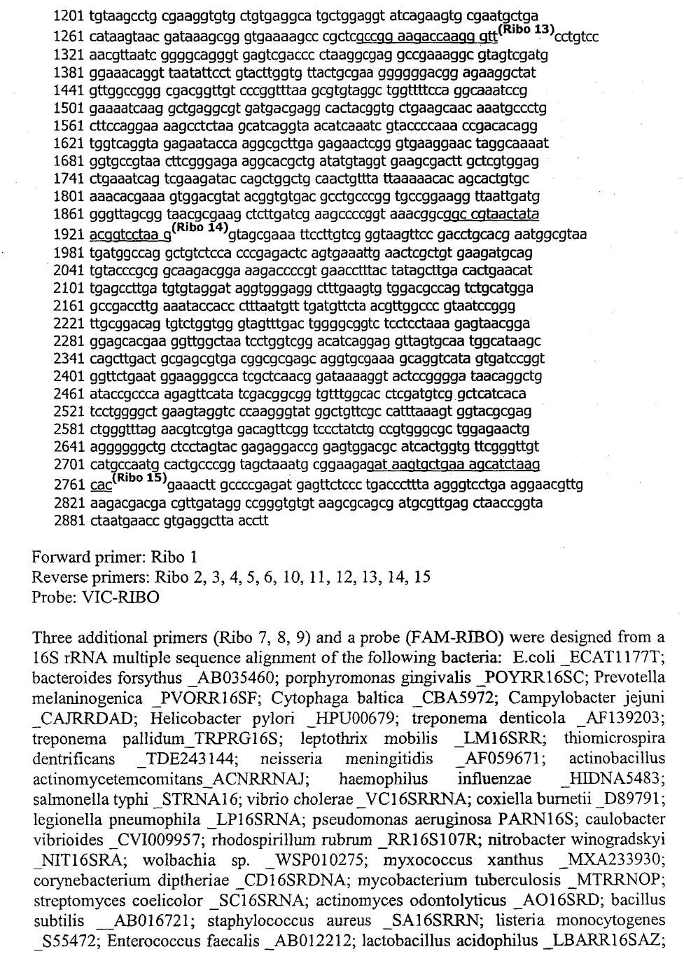

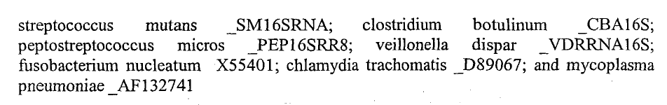

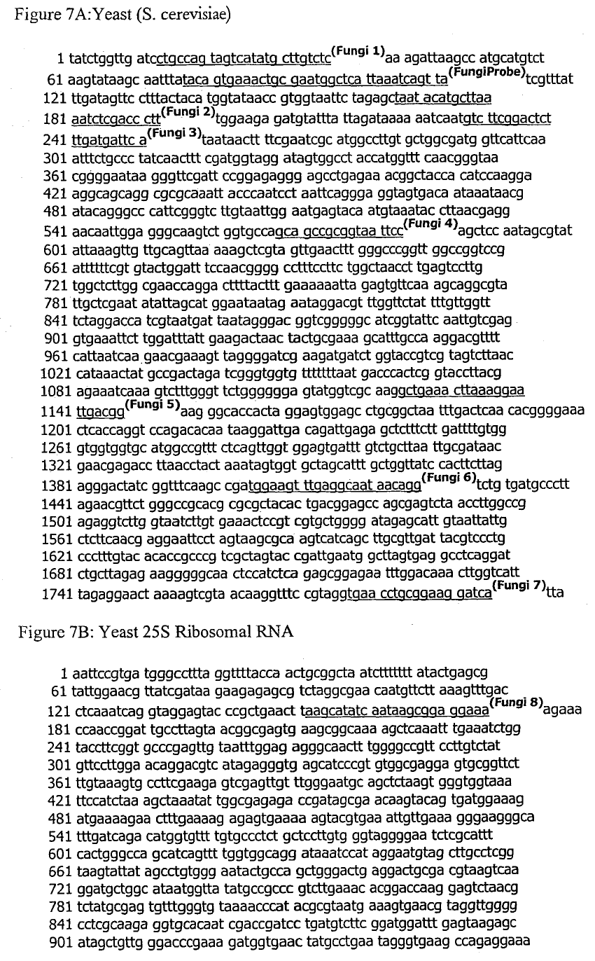

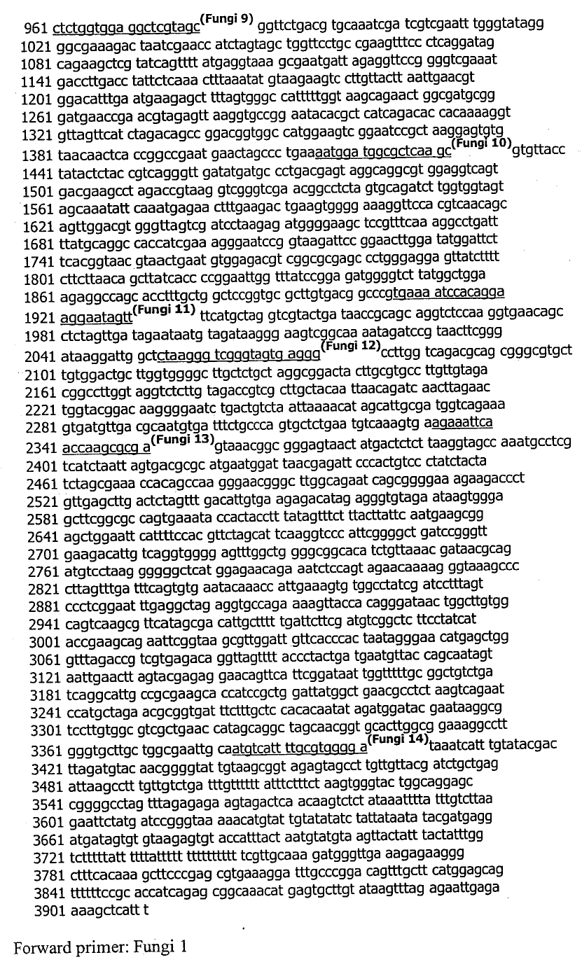

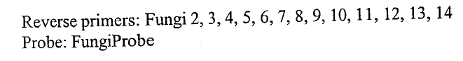

8. Primers: Ribo 7, 8, 10, 11, 12, 13, 14 and Fungi 1, 2, 3, 4, 5, 6, 7, 8 (FIGS. 6A and 6B);

9. Probes: FAM-RIBO

Fungi Probe 5′-6FAM-TACAGTGAAACTGCGAATGGCTCATTAAATCAGTTA-TAMRA-3′;

10. Microcon YM Centrifugal Filter Unit;

Procedure: 1. Using 0.05 tendon samples for E. coli and c. albicans, followed the Qiagen extraction profile;

2. Prepared the following master mixes:

| | Mix 1 | Mix 2 |
|---|---|---|
| 1 × buffer | 150 μl | 85 μl |
| dNTPs | 45 μl | 25.5 μl |
| Ribo 741.7 | — | |
| Fungi 1 | — | 23.65 μl |
| Taq | 30 μl | 17 μl |
| ProofStart | 30 μl | 17 μl |
| Water | 936.6 μl | 530.74 μl |
| FAM-RIBO | 75 μl | — |
| Fungi Probe | — | 42.5 μl |

3. Filtered master mixes using Microcon filter units for 30 minutes at 100×g;

4. Pipetted 43.6 μl of Mix 1 into: tows A-D, columns 1-6; tows A-C, column 9; and row E, column 12;

5. Pipetted 43.6 μl of Mix 2 into: rows E-F, columns 1-7 and row H, column 12;

6. Pipetted 1.39 μl of reverse primer into appropriate well;

7. Pipetted 5 μl DNA into appropriate wells;

8. Ran PCR.

Results: Irradiation with 50 kGy resulted in decreased amplification of large target amplicons, indicating degradation of the pathogen genetic material caused by irradiation.

### Example 17

Purpose: To show functionality of E. coli primers for RT-PCR using large target amplicons.

Materials: 1. E. coli prepared from overnight culture;

2. Dneasy Tissue Kit (Qiagen, cat. no. 96504);

3. Taq PCR Core Kit (Qiagen, cat. no. 201223)

4. ProofStart DNA polymerase (Qiagen, cat. no. 202205);

5. Microcon YM-100 Centrifugal Filter Unit (cat. no. 42413);

6. Primers: Ribo 1-9;

7. Agarose;

8. TAE Buffer;

9. Ethidium Bromide.

Procedure: 1. Pipetted 1 ml of E. coli culture into each of 10 1.5 tubes;

2. Centrifuged all 10 tubes for 5 minutes at maximum speed;

3. Discarded supernatant;

4. Placed 8 tubes in −80° C. and used 2 tubes for extraction following the Qiagen protocol;

5. Prepared Master Mix as follows:

10× Buffer 5 μl

dNTPs 1.5 μl

pA 1.39 μl (Ribo 1 or 7)

pB 1.39 μl (Ribo 2, 3, 4, 5, 6, 8 or 9)

Taq 1 μl

ProofStart 1 μl

Water 33.22 μl

TSP 0.5 μl

6. Mixed Master Mix by inversion;

7. Pipetted Master mix into a Microcon Centrifugal Filter Unit and centrifuged for 30 minutes at 100×g;

8. Pipetted 43.61 μl of Master Mix into PCR tubes;

9. Added appropriate reverse primer and DNA or water to create the following primer pairs: (1, 2)+5 μl DNA; (1, 2)+1 μl; (1, 3)+5 μl DNA; (1, 3)+1 μl DNA; (1, 4)+5 μl DNA; (1, 4)+1 μl DNA; (1, 5)+5 μl DNA; (1, 5)+1 μl DNA; (1, 6)+5 μl DNA; (1, 6)+1 μl DNA; (5, 8)+5 μl DNA; (7, 8)+1 μl DNA; (7, 9)+5 μl DNA; (7, 9)+1 μl DNA; and (1, 2)+5 (1, 4)+5 μl water;

10. Ran PCR;

11. Ran 1% Agarose gel at 100 V for about 20 min.

Results: All E. coli primers showed amplification of target sequences, regardless of size.

### Example 18

Purpose: To investigate the effects of 50 kGy irradiation on samples containing E. coli.

Materials: 1. E. coli spiked tendon (irradiated to 0 kGy and 50 kGy)+6.65×10¹⁰ CFU/μl;

2. Taq PCR Core Kit (Qiagen, cat. no. 201223);

3. ProofStart Taq Polymerase (Qiagen, cat. no. 202205);

4. Primers: Ribo 7, 8, 13, 14 and 15; 5. Agarose;

6. TAE Buffer;

7. Ethidium Bromide;

8. Microcon Centrifugal Filter Unit.

Procedure: 1. Prepared Master Mix as follows:

10× Buffer 60 μl

dNTP 18 μl

pA (forward) 16.68 μl

Taq 12 μl

ProofStart 12 μl

Water 452.64 μl;

2. Placed in Microcon and centrifuged for 30 minutes at 100×g;

3. Pipetted 47-61 μl master mix into each or 9 PCR tubes;

4. Added 1.39 μl of reverse primer and 1 μl DNA into appropriate tubes;

5. Ran PCR.

6. Ran 1% Agarose gel (8 μl sample+1 μl sample buffet) at 100 V for about 20 minutes.

Results: Samples irradiated to 50 kGy showed progessive disappearance of bands with increasing amplicon size, indicating degradation of the E. coli genetic material caused by irradiation.

### Example 19

Purpose: To show functionality of Mt-DNA primers for RT-PCR using large target amplicons.

Materials: 1. Tendon DNA (irradiated to 0 kGy and 50 kGy);

2. ROX 6 (1/10 dilution) molecular probes;

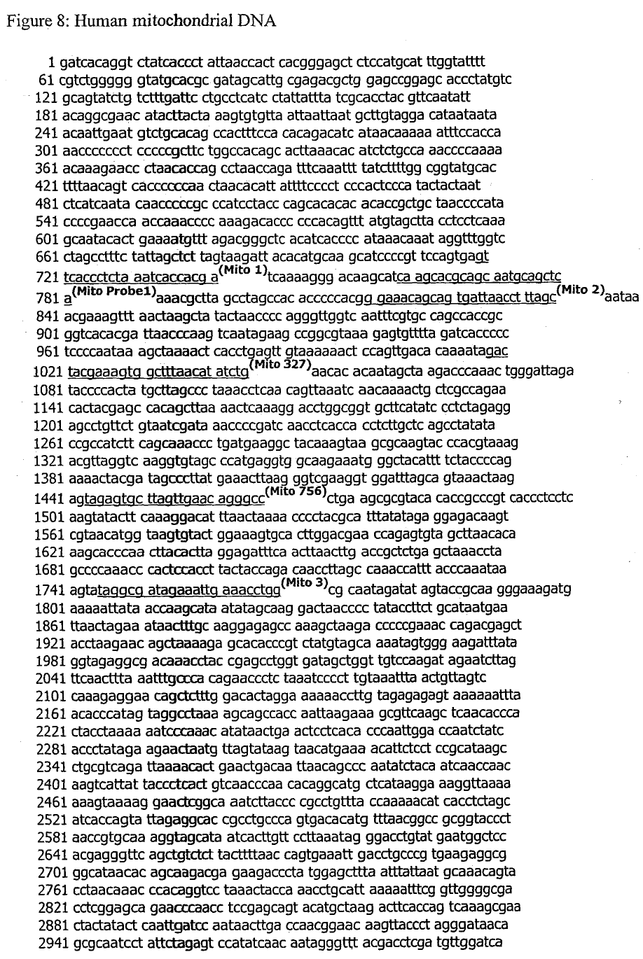

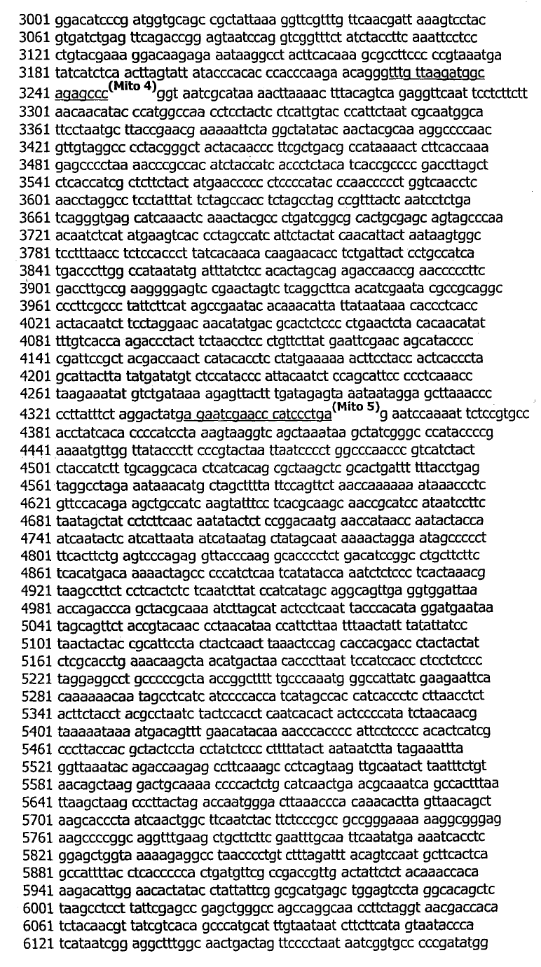

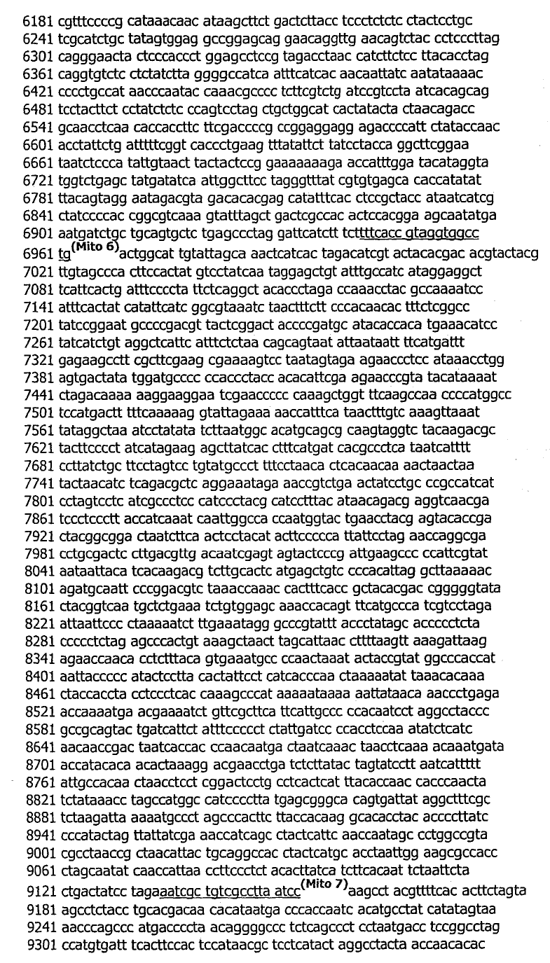

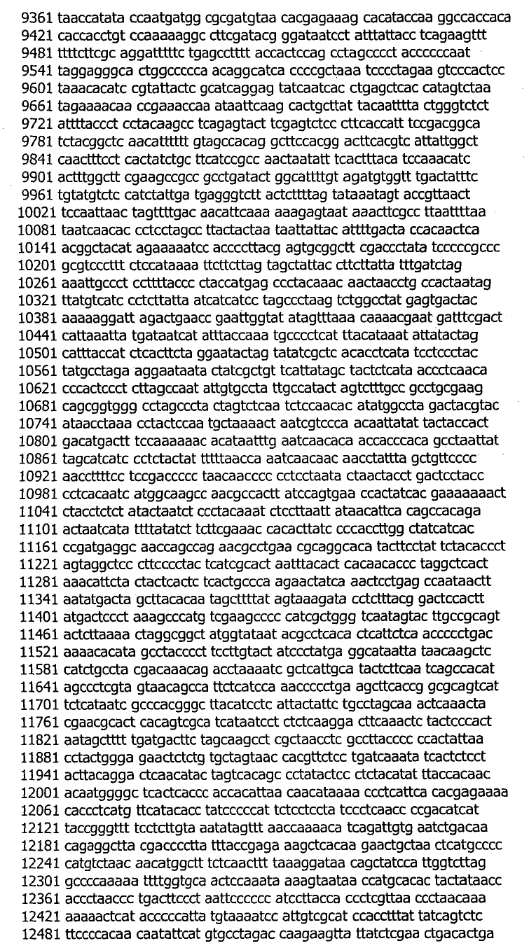

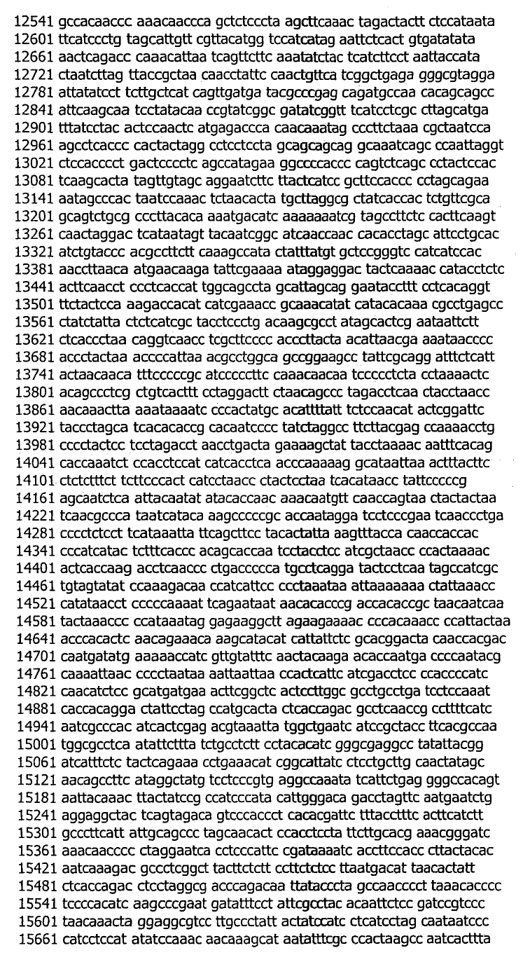

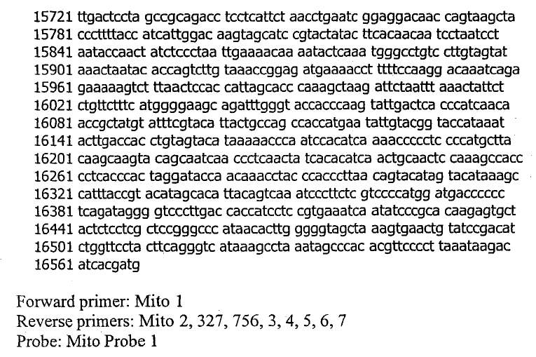

3. Primers: MITO 1, 2, 3, 4, and 5 (FIG. 8);

4. MITO Probe 1 (FIG. 8);

5. Human DNA;

6. Qiagen PCR Reagants;

7. Qiagen ProofStart.

Procedure: 1. Prepared the following mixtures:

Buffer 1.5 μl

dNTPs 1.5 μl

MITO 1 2.5 μl

reverse primer 2.5 μl (MITO 2, 3, 4 or 5)

MITO Probe 2.5 μl

Taq 1 μl

PS 1 μl

1/10 ROX 1 μl

water 28 μl

DNA 5 μl

2. Ran 40 PCR;

3. Ran 1% agarose gel (8 μl product+1 μl sample loading buffer) at 100 V for about one hour.

Results: Mt-DNA primers were functional, regardless of amplicon size.

Having now fully described this invention, it will be understood to those of ordinary skill in the art that the methods of the present invention can be carried out with a wide and equivalent range of conditions, formulations and other parameters without departing from the scope of the invention or any embodiments thereof.

All patents and publications cited herein are hereby fully incorporated by reference in their entirety. The citation of any publication is for its disclosure prior to the filing date and should not be construed as an admission that such publication is prior art or that the present invention is not entitled to antedate such publication by virtue of prior invention.

The foregoing embodiments and advantages are merely exemplary and are not to be construed as limiting the present invention. The present teaching can be readily applied to other types of apparatuses. The description of the present invention is intended to be illustrative, and not to limit the scope of the claims. Many alternatives, modifications, and variations will be apparent to those skilled in the art. In the claims, means-plus-function clauses are intended to cover the structures described herein as performing the recited function and not only structural equivalents but also equivalent structures.

What is claimed is:

1. A method for analyzing a target nucleic acid sequence in a biological material, said method comprising:
(i) adding to said biological material an effective amount of at least two nucleic acid primers,
wherein said nucleic acid primers hybridize under stringent conditions to predetermined nucleic acid sequences of said target nucleic acid sequence that are separated by at least about 750 nucleic acid residues,
(ii) amplifying said target nucleic acid sequence by polymerase chain reaction, said polymerase chain reaction comprising adding a polymerase and to said biological material and primers to form an amplification mixture and thermally cycling said amplification mixture between at least one denaturation temperature and at least one elongation temperature,
wherein said elongation temperature is not more than about 70□ C and said denaturation temperature is not more than about 95□ C, and further wherein during each thermal cycle said amplification mixture is maintained at said denaturation temperature for a period of not more than about 30 seconds and at said elongation temperature for a period of not less than about 1 minute; and
(iii) detecting and quantifying said target nucleic acid sequence.

2. The method according to claim 1, wherein said predetermined nucleic acid sequences of said target nucleic acid sequence are separated by at least about 1000 nucleic acid residues of said target nucleic acid sequence

3. The method according to claim 1, wherein said predetermined nucleic acid sequences of said target nucleic acid sequence are separated by at least about 2000 nucleic acid residues of said target nucleic acid sequence

4. The method according to claim 1, wherein said predetermined nucleic acid sequences of said target nucleic acid sequence are separated by at least about 3000 nucleic acid residues of said target nucleic acid sequence

5. The method according to claim 1, wherein said predetermined nucleic acid sequences of said target nucleic acid sequence are separated by at least about 4000 nucleic acid residues of said target nucleic acid sequence.

6. The method according to claim 1, wherein said predetermined nucleic acid sequences of said target nucleic acid sequence are separated by at least about 5000 nucleic acid residues of said target nucleic acid sequence.

7. The method according to claim 1, wherein said predetermined nucleic acid sequences of said target nucleic acid sequence are separated by only at least about 500 nucleic acid residues of said target nucleic acid sequence.

8. The method according to claim 1, wherein said step (i) further comprises adding at least one nucleic acid probe to said biological material.

9. The method according to claim 8, wherein said nucleic acid probe is selected from the group consisting of 5′ nuclease probes, hairpin probes, adjacent probes, sunrise probes and scorpion probes.

10. The method according to claim 1, wherein said elongation temperature is not more than about 70° C.

11. The method according to claim 1, wherein said elongation temperature is between about 60° C. and about 69° C.

12. The method according to claim 1, wherein said elongation temperature is between about 65° C. and about 69° C.

13. The method according to claim 1, wherein said denaturation temperature is not more than about 95° C.

14. The method according to claim 1, wherein said denaturation temperature is between about 90° C. and about 95° C.

15. The method according to claim 1, wherein said denaturation temperature is between about 93° C. and about 95° C.

16. The method according to claim 1, wherein during each thermal cycle said amplification mixture is maintained at said denaturation temperature for a period of not more than about 20 seconds.

17. The method according to claim 1, wherein during each thermal cycle said amplification mixture is maintained at said denaturation temperature for a period of not more than about 10 seconds.

18. The method according to claim 1, wherein during each thermal cycle said amplification mixture is maintained at said elongation temperature for a period of not less than about 2 minutes.

19. The method according to claim 1, wherein during each thermal cycle said amplification mixture is maintained at said elongation temperature for a period of not less than about 3 minutes.

20. The method according to claim 1, wherein the period during which said amplification mixture is maintained at said elongation temperature during each thermal cycle is increased by a period of about 5 seconds for each successive thermal cycle.

21. The method according to claim 1, wherein said amplification mixture is thermally cycled for at least 30 cycles.

22. The method according to claim 1, wherein said amplification mixture is thermally cycled for at least 40 cycles.

23. The method according to claim 1, wherein said amplification mixture is thermally cycled for at least 50 cycles.

24. The method according to claim 1, wherein said biological material has been subjected to an environment or process that may have altered said target nucleic acid sequence.

25. The method according to claim 1, wherein said thermostable polymerase is a Taq polymerase.

26. The method according to claim 1, wherein said thermostable polymerase is a proof-reading Taq polymerase.

27. The method according to claim 1, wherein said amplification mixture further comprises at least one thermostable inorganic pyrophosphatase.

28. The method according to claim 27, wherein the ratio of Taq polymerase to thermostable inorganic pyrophosphatase is about 5:1.


SEQUENCE LISTING (99 sequences)

SEQ ID NO: 1 - DNA, 5594 bp, B19 virus
ccaaatcagatgccgccggtcgccgccggtaggcgggacttccggtacaagatggcggacaattacgtcatttcctgtgacgtcatttcctgtgacgtcacttccggtgggcgggacttccggaattagggttggctctgggccagcttgcttggggttgccttgacactaagacaagcggcgcgccgcttgtcttagtggcacgtcaaccccaagcgctggcccagagccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttggtgtcttcttttaaattttagcgggcttttttcccgccttatgcaaatgggcagccattttaagtgtttcactataattttattggtcagttttgtaacggttaaaatgggcggagcgtaggcggggactacagtatatatagcacggcactgccgcagctctttctttctgggctgctttttcctggactttcttgctgttttttgtgagctaactaacaggtatttatactacttgttaacatactaacatggagctatttagaggggtgcttcaagtttcttctaatgttctggactgtgctaacgataactggtggtgctctttactggatttagacacttctgactgggaaccactaactcatactaacagactaatggcaatatacttaagcagtgtggcttctaagcttgactttaccggggggccactagcggggtgcttgtacttttttcaagtagaatgtaacaaatttgaagaaggctatcatattcatgtggttattggggggccagggttaaaccccagaaacctcacagtgtgtgtagaggggttatttaataatgtactttatcaccttgtaactgaaaatgtaaagctaaaatttttgccaggaatgactacaaaaggcaaatactttagagatggagagcagtttatagaaaactatttaatgaaaaaaatacctttaaatgttgtatggtgtgttactaatattgatggatatatagatacctgtatttctgctacttttagaaggggagcttgccatgccaagaaaccccgcattaccacagccataaatgacactagtagtgatgctggggagtctagcggcacaggggcagaggttgtgccaattaatgggaagggaactaaggctagcataaagtttcaaactatggtaaactggttgtgtgaaaacagagtgtttacagaggataagtggaaactagttgactttaaccagtacactttactaagcagtagtcacagtggaagttttcaaattcaaagtgcactaaaactagcaatttataaagcaactaatttagtgcctacaagcacatttctattgcatacagactttgagcaggttatgtgtattaaagacaataaaattgttaaattgttactttgtcaaaactatgaccccctattagtggggcagcatgtgttaaagtggattgataaaaaatgtggcaagaaaaatacactgtggttttatgggccgccaagtacaggaaaaacaaacttggcaatggccattgctaaaagtgttccagtatatggcatggttaactggaataatgaaaactttccatttaatgatgtagcagggaaaagcttggtggtctgggatgaaggtattattaagtctacaattgtagaagctgcaaaagccattttaggcgggcaacccaccagggtagatcaaaaaatgcgtggaagtgtagctgtgcctggagtacctgtggttataaccagcaatggtgacattacttttgttgtaagcgggaacactacaacaactgtacatgctaaagccttaaaagagcgaatggtaaagttaaactttactgtaagatgcagccctgacatggggttactaacagaggctgatgtacaacagtggcttacatggtgtaatgcacaaagctgggaccactatgaaaactgggcaataaactacacttttgatttccctggaattaatgcagatgccctccacccagacctccaaaccaccccaattgtcacagacaccagtatcagcagcagtggtggtgaaagctctgaagaactcagtgaaagcagcttttttaacctcatcaccccaggcgcctggaacactgaaaccccgcgctctagtacgcccatccccgggaccagttcaggagaatcatttgtcggaagctcagtttcctccgaagttgtagctgcatcgtgggaagaagccttctacacacctttggcagaccagtttcgtgaactgttagttggggttgattatgtgtgggacggtgtaaggggtttacctgtgtgttgtgtgcaacatattaacaatagtgggggaggcttgggactttgtccccattgcattaatgtaggggcttggtataatggatggaaatttcgagaatttaccccagatttggtgcggtgtagctgccatgtgggagcttctaatcccttttctgtgctaacctgcaaaaaatgtgcttacctgtctggattgcaaagctttgtagattatgagtaaagaaagtggcaaatggtgggaaagtgatgataaatttgctaaagctgtgtatcagcaatttgtggaattttatgaaaaggttactggaacagacttagagcttattcaaatattaaaagatcactataatatttctttagataatcccctagaaaacccatcctctctgtttgacttagttgctcgtattaaaaataaccttaaaaactctccagacttatatagtcatcattttcaaagtcatggacagttatctgaccacccccatgccttatcatccagtagcagtcatgcagaacctagaggagaaaatgcagtattatctagtgaagacttacacaagcctgggcaagttagcgtacaactacccggtactaactatgttgggcctggcaatgagctacaagctgggcccccgcaaagtgctgttgacagtgctgcaaggattcatgactttaggtatagccaactggctaagttgggaataaatccatatactcattggactgtagcagatgaagagcttttaaaaaatataaaaaatgaaactgggtttcaagcacaagtagtaaaagactactttactttaaaaggtgcagctgcccctgtggcccattttcaaggaagtttgccggaagttcccgcttacaacgcctcagaaaaatacccaagcatgacttcagttaattctgcagaagccagcactggtgcaggagggggtggcagtaatcctgtcaaaagcatgtggagtgagggggccacttttagtgccaactctgtaacttgtacattttccagacagtttttaattccttatgacccagagcaccattataaggtgttttctcccgcagcaagcagctgccacaatgccagtggaaaggaggcaaaggtttgcacaattagtcccataatgggatactcaaccccatggagatatttagattttaatgctttaaatttatttttttcacctttagagtttcagcacttaattgaaaattatggaagtatagctcctgatgctttaactgtaaccatatcagaaattgctgttaaggatgttacagacaaaactggagggggggtacaggttactgacagcactacagggcgcctatccatgttagtagaccatgaatacaagtacccatatgtgttaggacaaggtcaggatactttagccccagaacttcctatttgggtatactttccccctcaatatgcttacttaacagtaggagatgttaacacacaaggaatctctggagacagcaaaaaattagcaagtgaagaatcagcattttatgttttggaacacagttcttttcagcttttaggtacaggaggtacagcaactatgtcttataagtttcctccagtgcccccagaaaatttagagggctgcagtcaacacttttatgaaatgtacaatcccttatacggatcccgcttaggggttcctgacacattaggaggtgacccaaaatttagatctttaacacatgaagaccatgcaattcagccccaaaacttcatgccagggccactagtaaactcagtgtctacaaaggagggagacagctctaatactggagctggaaaagccttaacaggccttagcacaggcacctctcaaaacactagaatatccttacgccctgggccagtgtcacagccataccaccactgggacacagataaatatgttccaggaataaatgccatttctcatggtcagaccacttatggtaacgctgaagacaaagagtatcagcaaggagtgggtagatttccaaatgaaaaagaacagctaaaacagttacagggtttaaacatgcacacctatttccccaataaaggaacccagcaatatacagatcaaattgagcgccccctaatggtgggttctgtatggaacagaagagcccttcactatgaaagccagctgtggagtaaaattccaaatttagatgacagttttaaaactcagtttgcagccttaggaggatggggtttgcatcagccacctcctcaaatatttttaaaaatattaccacaaagtgggccaattggaggtattaaatcaatgggaattactaccttagttcagtatgccgtgggaattatgacagtaactatgacatttaaattggggccccgtaaagctacgggacggtggaatcctcaacctggagtatatcccccgcacgcagcaggtcatttaccatatgtactatatgaccccacagctacagatgcaaaacaacaccacaggcatggatacgaaaagcctgaagaattgtggacagccaaaagccgtgtgcacccattgtaaacactccccaccgtgccctcagccaggatgcgtaactaaacgcccaccagtaccacccagactgtacctgccccctcctgtacctataagacagcctaacacaaaagatatagacaatgtagaatttaagtacttaaccagatatgaacaacatgttattagaatgttaagattgtgtaatatgtatcaaaatttagaaaaataaacatttgttgtggttaaaaaattatgttgttgcgctttaaaaatttaaaagaagacaccaaatcagatgccgccggtcgccgccggtaggcgggacttccggtacaagatggcggacaattacgtcatttcctgtgacgtcatttcctgtgacgtcacttccggtgggcgggacttccggaattagggttggctctgggccagcgcttggggttgacgtgccactaagacaagcggcgcgccgcttgtcttagtgtcaaggcaaccccaagcaagctggcccagagccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttgg

SEQ ID NO: 2 - DNA, 3221 bp, Hepatitis B virus
ttccactgccttccaccaagctctgcaagaccccagagtcaggggtctgtattttcctgctggtggctccagttcaggaacagtaaaccctgctccgaatattgcctctcacatctcgtcaatctccgcgaggaccggggaccctgtgacgaacatggagaacatcacatcaggattcctaggacccctgcccgtgttacaggcggggtttttcttgttgacaagaatcctcacaataccgcagagtctagactcgtggtggacttctctcaattttctagggggatcacccgtgtgtcttggccaaaattcgcgatccccaacctccaatcactcaccaacctcctgtcctccaatttgtcctggttatcgctggatgtgtctgcggcgttttatcatattcctcttcatcctgctgctatgcctcatcttcttattggttcttctggattatcaaggtatgttgcccgtttgtcctctaattctaggatcaacaacaaccagtacgggaccatgcaaaacctgcacgactcctgctcaaggcaactctatgtttccctcatgttgctgtacaaaacctacggatggaaattgcacctgtattcccatcccatcgtcttgggctttcgcaaaatacctatgggagtgggcctcagtccgtttctcttggctcagtttactagtgccatttgttcagtggttcgtagggctttcccccactgtttggctttcagctatatggatgatgtggtattgggggccaagtctgtacagcatcgtgagttcctttataccgctgttaccaattttcttttgtctctgggtatacatttaaaccctaacaaaacaaaaagatggggttattccctaaacttcatgggttatgtaattggaagttggggaacattgccacaggatcatattgtacaaaaaatcaaacactgttttagaaaacttcctgttaacaggcctattgattggaaagtatgtcaaagaattgtgggtcttttgggctttgctgctccttttacacaatgtggatatcctgccttaatgcccttgtatgcatgtatacaagctaaacaggctttcactttctcgccaacttacaaggcctttctaagtaaacagtacatgaacctttaccccgttgctcggcaacggcctggtctgtgccaagtatttgctgatgcaacccccactggctggggcttggccataggccatcagcgcatgcgcggaacctttgtggctcctctgccgatccatactgcggaactcctagccgcttgttttgctcgcagccggtctggagcgaaactcatcggaactgacaattctgtcgtcctctcgcggaaatatacctcgtttccatggctactaggctgtgctgccaactggatccttcgcgggacgtcctttgtttacgtcccgtcggcgctgaatcccgcggacgacccctctcggggccgcttgggactctctcgtccccttctccgtctgccgttccagccgaccacggggcgcacctctctttacgcggtctccccgtctgtgccttctcatctgccggtccgtgtgcacttcgcttcacctctgcacgttgcatggagaccaccgtgaacgcccatcagatcctgcccaaggtcttacataagaggactcttggactcccagcaatgtcaacgaccgaccttgaggcctacttcaaagactgtgtgtttaaggactgggaggagctgggggaggagattaggttaaaggtctttgtattaggaggctgtaggcataaattggtctgcgcaccagcaccatgcaactttttcacctctgcctaatcatctcttgtacatgtcccactgttcaagcctccaagctgtgccttgggtggctttggggcatggacattgacccttataaagaatttggagctactgtggagttactctcgtttttgccttctgacttctttccttccgtcagagatctcctagacaccgcctcggctctgtatcgggaagccttagagtctcctgagcattgctcacctcaccataccgcactcaggcaagccattctctgctggggggaattgatgactctagctacctgggtgggtaataatttggaagatccagcatccagggatctagtagtcaattatgttaatactaacatgggattaaagatcaggcaactcttgtggtttcatatctcttgccttacttttggaagagaaactgtacttgaatatttggtctctttcggagtgtggattcgcactcctccagcctatagaccaccaaatgcccctatcttatcaacacttccggaaactactgttgttagacgacgggaccgaggcaggtcccctagaagaagaactccctcgcctcgcagacgcagatctcaatcgccgcgtcgcagaagatctcaatctcgggaatctcaatgttagtattccttggactcataaggtgggaaacttcactgggctttattcctctacagcacctatctttaatcctgaatggcaaactccttcctttcctaaaattcatttacaagaggacattattaataggtgtcaacaatttgtgggccctctcactgtaaatgaaaagagaagattgaaattaattatgcctgctagattctatcctacccacactaaatatttgcccttagacaaaggaattaaaccttattatccagatcaggtagttaatcattacttccaaaccagacattatttacatactctttggaaggcgggtattctatataagagagaaaccacacgtagcgcatcattttgcgggtcaccatattcttgggaacaagagctacagcatgggaggttggtcatcaaaacctcgcaaaggcatggggacgaatctttctgttcccaaccctctgggattctttcccgatcatcagttggaccctgtattcggagccaactcaaacaatccagattgggacttcaaccccatcaaggaccactggccagcagccaaccaggtaggagtgggagcattcgggccagggttcacccctccacacggcggtgttttggggtggagccctcaggctcagggcatgttgaccccagtgtcaacaattcctcctcctgcctccgccaatcggcagtcaggaaggcagcctactcccatctctccacctctaagagacagtcatcctcaggccatgcagtggaa

SEQ ID NO: 3 - DNA, 5075 bp, Porcine parvovirus
aatctttaaactgaccaactgtctttgcgtatggtgacgtgatgacgcgcgctacgcgcgctgccttcggcagtcacacgtcaccatcagcaaagacagttggtcagtttaaagattaataagacattccattggctgaaaagaggcgggaaattcaaaaaaagaggcgggaaaaaaagaggtggagcctaacactataaatacagttgcttacttcagttagttcctttctgcttcagactgcacttcgctccagagacacagctacaaactactctcagctactgcagcatggcagcgggaaacacttactcggaagaggtactaaaagctaccaactggcttcaagataatgctcaaaaagaagcattctcttatgtatttaaaacacaaaaagtcaatctaaatggaaaagaaattgcttggaataactacaacaaagatacaacagatgcggaaatgataaacctacaaagaggagcagaaacatcatgggaccaggcaacagacatggaatgggaatcagaaatcgacagcctcacaaaacggcaagtactgatttttgactctcttgttaaaaaatgtctctttgaaggtatattgcaaaagaacctaagtccaagtgactgctactggttcatacagcatgaacatggtcaagatactggctatcactgccatgtactactaggtggaaaaggcttacaacaagcaatgggaaaatggttcagaaaacaattaaacaatttatggagtagatggttaataatgcaatgcaaagtacctctaacaccagttgaaagaataaaattaagggaattagcagaggatggtgagtgggtatcgctactaacctacactcacaaacaaactaaaaaacaatatacaaaaatgactcattttggaaatatgattgcttactacttcctaaataaaaaaagaaagacaactgaaagagagcatggatattatctcagctcagattctggcttcatgacaaatttcttaaaagaaggcgagagacacttagtcagtcacctatttactgaagcaaataaacctgaaactgtggaaacaacggttactacagctcaggaagccaaaagaggcagaatacaaacaaaaaaagaagtaagcataaaatgcacaataagagacttggttaataaaagatgtactagcatagaagactggatgatgacagatccagacagttatatagaaatgatggctcaaaccggaggagaaaatttaatcaaaaatacactagaaataacaactcttactctagcaagaacaaaaacagcatatgacttaatacttgaaaaggcaaaaccaagcatgctaccaacatttaatattagcaatacaagaacatgtaaaatattcagcatgcacaattggaactacattaaagtctgccatgctataacttgtgtactaaacagacaaggaggaaaaagaaatacaattctatttcatgggccagcatcaacaggaaaaagtataattgctcaacacattgcaaacttagttggtaatgttggttgctacaatgcagccaatgtgaactttccatttaatgactgtacaaataaaaacttaatatggattgaagaagcaggaaacttctctaaccaagtaaaccaattcaaagccatatgttcaggtcaaacaattagaattgaccaaaaaggtaaaggaagcaaacaaattgaaccaactcctgtaataatgactacaaatgaagacataactaaagttagaataggatgcgaggaaagaccagaacatacacaaccaataagagacagaatgttaaacataaacctaaccagaaaactgccaggtgattttggacttttagaagaaactgaatggccactaatatgtgcttggttggtaaagaaaggttaccaagcaacaatggctagctatatgcatcattggggaaatgtacctgattggtcagaaaaatgggaggagccaaaaatgcaaaccccaataaatacaccaacagactctcagatttccacatcagtgaaaacttcgccagcggacaacaactacgcagcaactccaatacaggaggacctggatttagctttagccttggagccgtggagcgagccaacaacaccaactttcaccaacctgcacttaactccaacaccgccagattcagcaatacggacaccaagtccaacttggtcggaaatagaaaccgacataagagcctgctttggtgaaaactgtgcacccacaacaaaccttgaataaggtaggatggcgcctcctgcaaaaagagcaagaggtaagggtagttttaagggggtggtgggcatacatataaaactaactgcaaataatttttttatatattacaggactaactctaccaggatacaaataccttggtccaggaaactcactagaccaaggagaaccaactaatccatcagacgccgcagcaaaagaacacgacgaagcctacgacaaatacataaaatctggaaaaaatccatacttctacttctcagcagctgatgaaaaattcataaaagaaactgaacacgcaaaagactacggaggtaaaattggacattacttcttcagagcaaagcgtgcctttgctccaaaactctcagaaacagactcaccaactacatctcaacaaccagaggtaagaagatcgccgagaaaacacccagggtctaaaccaccaggaaaaagacctgctccaagacatatttttataaacttagctaaaaaaaaagctaaagggacatctaatacaaactctaactcaatgagtgaaaatgtggaacaacacaaccctattaatgcaggcactgaattgtctgcaacaggaaatgaatctgggggtgggggcggcggtggcgggggtaggggtgctgggggggttggtgtgtctacaggtactttcaataatcaaacagaatttcaatacttgggggagggcttggttagaatcactgcacacgcatcaagactcatacatctaaatatgccagaacacgaaacatacaaaagaatacatgtactaaattcagaatcaggggtggcgggacaaatggtacaagacgatgcacacacacaaatggtaacaccttggtcactaatagatgctaacgcatggggagtgtggttcaatccagcggactggcagttaatatccaacaacatgacagaaataaacttagttagttttgaacaagaaatattcaatgtagtacttaaaacaattacagaatcagcaacctcaccaccaaccaaaatatataataatgatctaactgcaagcttaatggtcgcactagacaccaataacacacttccatacacaccagcagcacctagaagtgaaacacttggtttttatccatggttacctacaaaaccaactcaatacagatattacctatcatgcatcagaaacctaaatccaccaacatacactggacaatcacaacaaataacagactcaatacaaacaggactacacagtgacattatgttctacacaatagaaaatgcagtaccaattcatcttctaagaacaggagatgaattctccacaggaatatatcactttgacacaaaaccactaaaattaactcactcatggcaaacaaacagatctctaggactgcctccaaaactactaactgaacctaccacagaaggagaccaacacccaggaacactaccagcagctaacacaagaaaaggttatcaccaaacaattaataatagctacacagaagcaacagcaattaggccagctcaggtaggatataatacaccatacatgaattttgaatactccaatggtggaccatttctaactcctatagtaccaacagcagacacacaatataatgatgatgaaccaaatggtgctataagatttacaatggattaccaacatggacacttaaccacatcttcacaagagctagaaagatacacattcaatccacaaagtaaatgtggaagagctccaaagcaacaatttaatcaacaggcaccactaaacctagaaaatacaaataatggaacacttttaccttcagatccaataggagggaaatctaacatgcatttcatgaatacactcaatacatatggaccattaacagcactaaacaatactgcacctgtatttccaaatggtcaaatatgggataaagaacttgatacagatctaaaacctagactacatgttacagctccatttgtttgtaaaaacaatccaccaggacaactatttgtaaaaatagcaccaaacctaacagatgatttcaatgctgactctcctcaacaacctagaataataacttattcaaacttttggtggaaaggaacactaacattcacagcaaaaatgagatccagtaatatgtggaaccctattcaacaacacacaacaacagcagaaaacattggtaactatattcctacaaatattggtggcataagaatgtttccagaatattcacaacttataccaagaaaattatactagaaataactctgtaaataaaaactcagttacttggttaatcatgtactactatcattgtatacttcaataaaaataaattgtaaaatcaataaaactaagttacttagtttctgtatacctatactagaaataactctgtaaataaaaactcagttacttggttaatcatgtactactatcattgtatacttcaataaaaataaattgtaaaatcaataaaactaagttacttagtttctgtataccaattatccccaaaaaacaataaaattttaaaaagaaacaagctctcatgtgtttactattaactaaaccaaccacacttatatgaccttatgtctttagggtgggtgggtgggaattactatgtattcctttgagttagttggtcgcctttgggcgactaaccaagcggctctgccgcttggttagtcgcacggcgaccaactaactcaaaggaatacatagtaattcccacccacccaccctaaagacataaggtcatataagtgtggttggtttagtt

SEQ ID NO: 4 - DNA, 11703 bp, Sindbis virus
attgacggcgtagtacacactattgaatcaaacagccgaccaattgcactaccatcacaatggagaagccagtagtaaacgtagacgtagacccccagagtccgtttgtcgtgcaactgcaaaaaagcttcccgcaatttgaggtagtagcacagcaggtcactccaaatgaccatgctaatgccagagcattttcgcatctggccagtaaactaatcgagctggaggttcctaccacagcgacgatcttggacataggcagcgcaccggctcgtagaatgttttccgagcaccagtatcattgtgtctgccccatgcgtagtccagaagacccggaccgcatgatgaaatacgccagtaaactggcggaaaaagcgtgcaagattacaaacaagaacttgcatgagaagattaaggatctccggaccgtacttgatacgccggatgctgaaacaccatcgctctgctttcacaacgatgttacctgcaacatgcgtgccgaatattccgtcatgcaggacgtgtatatcaacgctcccggaactatctatcatcaggctatgaaaggcgtgcggaccctgtactggattggcttcgacaccacccagttcatgttctcggctatggcaggttcgtaccctgcgtacaacaccaactgggccgacgagaaagtccttgaagcgcgtaacatcggactttgcagcacaaagctgagtgaaggtaggacaggaaaattgtcgataatgaggaagaaggagttgaagcccgggtcgcgggtttatttctccgtaggatcgacactttatccagaacacagagccagcttgcagagctggcatcttccatcggtgttccacttgaatggaaagcagtcgtacacttgccgctgtgatacagtggtgagttgcgaaggctacgtagtgaagaaaatcaccatcagtcccgggatcacgggagaaaccgtgggatacgcggttacacacaatagcgagggcttcttgctatgcaaagttactgacacagtaaaaggagaacgggtatcgttccctgtgtgcacgtacatcccggccaccatatgcgatcagatgactggtataatggccacggatatatcacctgacgatgcacaaaaacttctggttgggctcaaccagcgaattgtcattaacggtaggactaacaggaacaccaacaccatgcaaaattaccttctgccgatcatagcacaagggttcagcaaatgggctaaggagcgcaaggatgatcttgataacgagaaaatgctgggtactagagaacgcaagcttacgtatggctgcttgtgggcgtttcgcactaagaaagtacattcgttttatcgcccacctggaacgcagacctgcgtaaaagtcccagcctcttttagcgcttttcccatgtcgtccgtatggacgacctctttgcccatgtcgctgaggcagaaattgaaactggcattgcaaccaaagaaggaggaaaaactgctgcaggtctcggaggaattagtcatggaggccaaggctgcttttgaggatgctcaggaggaagccagagcggagaagctccgagaagcacttccaccattagtggcagacaaaggcatcgaggcagccgcagaagttgtctgcgaagtggaggggctccaggcggacatcggagcagcattagttgaaaccccgcgcggtcacgtaaggataatacctcaagcaaatgaccgtatgatcggacagtatatcgttgtctcgccaaactctgtgctgaagaatgccaaactcgcaccagcgcacccgctagcagatcaggttaagatcataacacactccggaagatcaggaaggtacgcggtcgaaccatacgacgctaaagtactgatgccagcaggaggtgccgtaccatggccagaattcctagcactgagtgagagcgccacgttagtgtacaacgaaagagagtttgtgaaccgcaaactataccacattgccatgcatggccccgccaagaatacagaagaggagcagtacaaggttacaaaggcagagcttgcagaaacagagtacgtgtttgacgtggacaagaagcgttgcgttaagaaggaagaagcctcaggtctggtcctctcgggagaactgaccaaccctccctatcatgagctagctctggagggactgaagacccgacctgcggtcccgtacaaggtcgaaacaataggagtgataggcacaccggggtcgggcaagtcagctattatcaagtcaactgtcacggcacgagatcttgttaccagcggaaagaaagaaaattgtcgcgaaattgaggccgacgtgctaagactgaggggtatgcagattacgtcgaagacagtagattcggttatgctcaacggatgccacaaagccgtagaagtgctgtacgttgacgaagcgttcgcgtgccacgcaggagcactacttgccttgattgctatcgtcaggccccgcaagaaggtagtactatgcggagaccccatgcaatgcggattcttcaacatgatgcaactaaaggtacatttcaatcaccctgaaaaagacatatgcaccaagacattctacaagtatatctcccggcgttgcacacagccagttacagctattgtatcgacactgcattacgatggaaagatgaaaaccacgaacccgtgcaagaagaacattgaaatcgatattacaggggccacaaagccgaagccaggggatatcatcctgacatgtttccgcgggtgggttaagcaattgcaaatcgactatcccggacatgaagtaatgacagccgcggcctcacaagggctaaccagaaaaggagtgtatgccgtccggcaaaaagtcaatgaaaacccactgtacgcgatcacatcagagcatgtgaacgtgttgctcacccgcactgaggacaggctagtgtggaaaaccttgcagggcgacccatggattaagcagcccactaacatacctaaaggaaactttcaggctactatagaggactgggaagctgaacacaagggaataattgctgcaataaacagccccactccccgtgccaatccgttcagctgcaagaccaacgtttgctgggcgaaagcattggaaccgatactagccacggccggtatcgtacttaccggttgccagtggagcgaactgttcccacagtttgcggatgacaaaccacattcggccatttacgccttagacgtaatttgcattaagtttttcggcatggacttgacaagcggactgttttctaaacagagcatcccactaacgtaccatcccgccgattcagcgaggccggtagctcattgggacaacagcccaggaacccgcaagtatgggtacgatcacgccattgccgccgaactctcccgtagatttccggtgttccagctagctgggaagggcacacaacttgatttgcagacggggagaaccagagttatctctgcacagcataacctggtcccggtgaaccgcaatcttcctcacgccttagtccccgagtacaaggagaagcaacccggcccggtcaaaaaattcttgaaccagttcaaacaccactcagtacttgtggtatcagaggaaaaaattgaagctccccgtaagagaatcgaatggatcgccccgattggcatagccggtgcagataagaactacaacctggctttcgggtttccgccgcaggcacggtacgacctggtgttcatcaacattggaactaaatacagaaaccaccactttcagcagtgcgaagaccatgcggcgaccttaaaaaccctttcgcgttcggccctgaattgccttaacccaggaggcaccctcgtggtgaagtcctatggctacgccgaccgcaacagtgaggacgtagtcaccgctcttgccagaaagtttgtcagggtgtctgcagcgagaccagattgtgtctcaagcaatacagaaatgtacctgattttccgacaactagacaacagccgtacacggcaattcaccccgcaccatctgaattgcgtgatttcgtccgtgtatgagggtacaagagatggagttggagccgcgccgtcataccgcaccaaaagggagaatattgctgactgtcaagaggaagcagttgtcaacgcagccaatccgctgggtagaccaggcgaaggagtctgccgtgccatctataaacgttggccgaccagttttaccgattcagccacggagacaggcaccgcaagaatgactgtgtgcctaggaaagaaagtgatccacgcggtcggccctgatttccggaagcacccagaagcagaagccttgaaattgctacaaaacgcctaccatgcagtggcagacttagtaaatgaacataacatcaagtctgtcgccattccactgctatctacaggcatttacgcagccggaaaagaccgccttgaagtatcacttaactgcttgacaaccgcgctagacagaactgacgcggacgtaaccatctattgcctggataagaagtggaaggaaagaatcgacgcggcactccaacttaaggagtctgtaacagagctgaaggatgaagatatggagatcgacgatgagttagtatggatccatccagacagttgcttgaagggaagaaagggattcagtactacaaaaggaaaattgtattcgtacttcgaaggcaccaaattccatcaagcagcaaaagacatggcggagataaaggtcctgttccctaatgaccaggaaagtaatgaacaactgtgtgcctacatattgggtgagaccatggaagcaatccgcgaaaagtgcccggtcgaccataacccgtcgtctagcccgcccaaaacgttgccgtgcctttgcatgtatgccatgacgccagaaagggtccacagacttagaagcaataacgtcaaagaagttacagtatgctcctccaccccccttcctaagcacaaaattaagaatgttcagaaggttcagtgcacgaaagtagtcctgtttaatccgcacactcccgcattcgttcccgcccgtaagtacatagaagtgccagaacagcctaccgctcctcctgcacaggccgaggaggcccccgaagttgtagcgacaccgtcaccatctacagctgataacacctcgcttgatgtcacagacatctcactggatatggatgacagtagcgaaggctcacttttttcgagctttagcggatcggacaactctattactagtatggacagttggtcgtcaggacctagttcactagagatagtagaccgaaggcaggtggtggtggctgacgttcatgccgtccaagagcctgcccctattccaccgccaaggctaaagaagatggcccgcctggcagcggcaagaaaagagcccactccaccggcaagcaatagctctgagtccctccacctctcttttggtggggtatccatgtccctcggatcaattttcgacggagagacggcccgccaggcagcggtacaacccctggcaacaggccccacggatgtgcctatgtctttcggatcgttttccgacggagagattgatgagctgagccgcagagtaactgagtccgaacccgtcctgtttggatcatttgaaccgggcgaagtgaactcaattatatcgtcccgatcagccgtatcttttccactacgcaagcagagacgtagacgcaggagcaggaggactgaatactgactaaccggggtaggtgggtacatattttcgacggacacaggccctgggcacttgcaaaagaagtccgttctgcagaaccagcttacagaaccgaccttggagcgcaatgtcctggaaagaattcatgccccggtgctcgacacgtcgaaagaggaacaactcaaactcaggtaccagatgatgcccaccgaagccaacaaaagtaggtaccagtctcgtaaagtagaaaatcagaaagccataaccactgagcgactactgtcaggactacgactgtataactctgccacagatcagccagaatgctataagatcacctatccgaaaccattgtactccagtagcgtaccggcgaactactccgatccacagttcgctgtagctgtctgtaacaactatctgcatgagaactatccgacagtagcatcttatcagattactgacgagtacgatgcttacttggatatggtagacgggacagtcgcctgcctggatactgcaaccttctgccccgctaagcttagaagttacccgaaaaaacatgagtatagagccccgaatatccgcagtgcggttccatcagcgatgcagaacacgctacaaaatgtgctcattgccgcaactaaaagaaattgcaacgtcacgcagatgcgtgaactgccaacactggactcagcgacattcaatgtcgaatgctttcgaaaatatgcatgtaatgacgagtattgggaggagttcgctcggaagccaattaggattaccactgagtttgtcaccgcatatgtagctagactgaaaggccctaaggccgccgcactatttgcaaagacgtataatttggtcccattgcaagaagtgcctatggatagattcgtcatggacatgaaaagagacgtgaaagttacaccaggcacgaaacacacagaagaaagaccgaaagtacaagtgatacaagccgcagaacccctggcgactgcttacttatgcgggattcaccgggaattagtgcgtaggcttacggccgtcttgcttccaaacattcacacgctttttgacatgtcggcggaggattttgatgcaatcatagcagaacacttcaagcaaggcgacccggtactggagacggatatcgcatcattcgacaaaagccaagacgacgctatggcgttaaccggtctgatgatcttggaggacctgggtgtggatcaaccactactcgacttgatcgagtgcgcctttggagaaatatcatccacccatctacctacgggtactcgttttaaattcggggcgatgatgaaatccggaatgttcctcacactttttgtcaacacagttttgaatgtcgttatcgccagcagagtactagaagagcggcttaaaacgtccagatgtgcagcgttcattggcgacgacaacatcatacatggagtagtatctgacaaagaaatggctgagaggtgcgccacctggctcaacatggaggttaagatcatcgacgcagtcatcggtgagagaccaccttacttctgcggcggatttatcttgcaagattcggttacttccacagcgtgccgcgtggcggatcccctgaaaaggctgtttaagttgggtaaaccgctcccagccgacgacgagcaagacgaagacagaagacgcgctctgctagatgaaacaaaggcgtggtttagagtaggtataacaggcactttagcagtggccgtgacgacccggtatgaggtagacaatattacacctgtcctactggcattgagaacttttgcccagagcaaaagagcattccaagccatcagaggggaaataaagcatctctacggtggtcctaaatagtcagcatagtacatttcatctgactaatactacaacaccaccaccatgaatagaggattctttaacatgctcggccgccgccccttcccggcccccactgccatgtggaggccgcggagaaggaggcaggcggccccgatgcctgcccgcaacgggctggcttctcaaatccagcaactgaccacagccgtcagtgccctagtcattggacaggcaactagacctcaacccccacgtccacgcccgccaccgcgccagaagaagcaggcgcccaagcaaccaccgaagccgaagaaaccaaaaacgcaggagaagaagaagaagcaacctgcaaaacccaaacccggaaagagacagcgcatggcacttaagttggaggccgacagattgttcgacgtcaagaacgaggacggagatgtcatcgggcacgcactggccatggaaggaaaggtaatgaaacctctgcacgtgaaaggaaccatcgaccaccctgtgctatcaaagctcaaatttaccaagtcgtcagcatacgacatggagttcgcacagttgccagtcaacatgagaagtgaggcattcacctacaccagtgaacaccccgaaggattctataactggcaccacggagcggtgcagtatagtggaggtagatttaccatccctcgcggagtaggaggcagaggagacagcggtcgtccgatcatggataactccggtcgggttgtcgcgatagtcctcggtggcgctgatgaaggaacacgaactgccctttcggtcgtcacctggaatagtaaagggaagacaattaagacgaccccggaagggacagaagagtggtccgcagcaccactggtcacggcaatgtgtttgctcggaaatgtgagcttcccatgcgaccgcccgcccacatgctatacccgcgaaccttccagagccctcgacatccttgaagagaacgtgaaccatgaggcctacgataccctgctcaatgccatattgcggtgcggatcgtctggcagaagcaaaagaagcgtcattgacgactttaccctgaccagcccctacttgggcacatgctcgtactgccaccatactgtaccgtgcttcagccctgttaagatcgagcaggtctgggacgaagcggacgataacaccatacgcatacagacttccgcccagtttggatacgaccaaagcggagcagcaagcgcaaacaagtaccgctacatgtcgcttaagcaggatcacaccgttaaagaaggcaccatggatgacatcaagattagcacctcaggaccgtgtagaaggcttagctacaaaggatactttctcctcgcaaaatgccctccaggggacagcgtaacggttagcatagtgagtagcaactcagcaacgtcatgtacactggcccgcaagataaaaccaaaattcgtgggacgggaaaaatatgatctacctcccgttcacggtaaaaaaattccttgcacagtgtacgaccgtctgaaagaaacaactgcaggctacatcactatgcacaggccgagaccgcacgcttatacatcctacctggaagaatcatcagggaaagtttacgcaaagccgccatctgggaagaacattacgtatgagtgcaagtgcggcgactacaagaccggaaccgtttcgacccgcaccgaaatcactggttgcaccgccatcaagcagtgcgtcgcctataagagcgaccaaacgaagtgggtcttcaactcaccggacttgatcagacatgacgaccacacggcccaagggaaattgcatttgcctttcaagttgatcccgagtacctgcatggtccctgttgcccacgcgccgaatgtaatacatggctttaaacacatcagcctccaattagatacagaccacttgacattgctcaccaccaggagactaggggcaaacccggaaccaaccactgaatggatcgtcggaaagacggtcagaaacttcaccgtcgaccgagatggcctggaatacatatggggaaatcatgagccagtgagggtctatgcccaagagtcagcaccaggagaccctcacggatggccacacgaaatagtacagcattactaccatcgccatcctgtgtacaccatcttagccgtcgcatcagctaccgtggcgatgatgattggcgtaactgttgcagtgttatgtgcctgtaaagcgcgccgtgagtgcctgacgccatacgccctggccccaaacgccgtaatcccaacttcgctggcactcttgtgctgcgttaggtcggccaatgctgaaacgttcaccgagaccatgagttacttgtggtcgaacagtcagccgttcttctgggtccagttgtgcatacctttggccgctttcatcgttctaatgcgctgctgctcctgctgcctgccttttttagtggttgccggcgcctacctggcgaaggtagacgcctacgaacatgcgaccactgttccaaatgtgccacagataccgtataaggcacttgttgaaagggcagggtatgccccgctcaatttggagatcactgtcatgtcctcggaggttttgccttccaccaaccaagagtacattacctgcaaattcaccactgtggtcccctccccaaaaatcaaatgctgcggctccttggaatgtcagccggccgctcatgcagactatacctgcaaggtcttcggaggggtctacccctttatgtggggaggagcgcaatgtttttgcgacagtgagaacagccagatgagtgaggcgtacgtcgaattgtcagcagattgcgcgtctgaccacgcgcaggcgattaaggtgcacactgccgcgatgaaagtaggactgcgtattgtgtacgggaacactaccagtttcctagatgtgtacgtgaacggagtcacaccaggaacgtctaaagacttgaaagtcatagctggaccaatttcagcatcgtttacgccattcgatcataaggtcgttatccatcgcggcctggtgtacaactatgacttcccggaatatggagcgatgaaaccaggagcgtttggagacattcaagctacctccttgactagcaaggatctcatcgccagcacagacattaggctactcaagccttccgccaagaacgtgcatgtcccgtacacgcaggcctcatcaggatttgagatgtggaaaaacaactcaggccgcccactgcaggaaaccgcacctttcgggtgtaagattgcagtaaatccgctccgagcggtggactgttcatacgggaacattcccatttctattgacatcccgaacgctgcctttatcaggacatcagatgcaccactggtctcaacagtcaaatgtgaagtcagtgagtgcacttattcagcagacttcggcgggatggccaccctgcagtatgtatccgaccgcgaaggtcaatgccccgtacattcgcattcgagcacagcaactctccaagagtcgacagtacatgtcctggagaaaggagcggtgacagtacactttagcaccgcgagtccacaggcgaactttatcgtatcgctgtgtgggaagaagacaacatgcaatgcagaatgtaaaccaccagctgaccatatcgtgagcaccccgcacaaaaatgaccaagaatttcaagccgccatctcaaaaacatcatggagttggctgtttgcccttttcggcggcgcctcgtcgctattaattataggacttatgatttttgcttgcagcatgatgctgactagcacacgaagatgaccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttc

SEQ ID NO: 5 - DNA, 10945 bp, West Nile virus
gaggattaacaacaattaacacagtgcgagctgtttcttagcacgaagatctcgatgtctaagaaaccaggagggcccggcaagagccgggctgtcaatatgctaaaacgcggaatgccccgcgtgttgtccttgattggactgaagagggctatgttgagcctgatcgacggcaaggggccaatacgatttgtgttggctctcttggcgttcttcaggttcacagcaattgctccgacccgagcagtgctggatcgatggagaggtgtgaacaaacaaacagcgatgaaacaccttctgagttttaagaaggaactagggaccttgaccagtgctatcaatcggcggagctcaaaacaaaagaaaagaggaggaaagaccggaattgcagtcatgattggcctgatcgccagcgtaggagcagttaccctctctaacttccaagggaaggtgatgatgacggtaaatgctactgacgtcacagatgtcatcacgattccaacagctgctggaaagaacctatgcattgtcagagcaatggatgtgggatacatgtgcgatgatactatcacctatgaatgcccagtgctgtcggctggtaatgatccagaagacatcgactgttggtgcacaaagtcagcagtctacgtcaggtatggaagatgcaccaagacacgccactcaagacgcagtcggaggtcactgacagtgcagacacacggagaaagcactctagcgaacaagaagggggcttggatggacagcaccaaggccacaaggtatttggtaaaaacagaatcatggatcttgaggaaccctggatatgccctggtggcagccgtcattggttggatgcttgggagcaacaccatgcagagagttgtgtttgtcgtgctattgcttttggtggccccagcttacagcttcaactgccttggaatgagcaacagagacttcttggaaggagtgtctggagcaacatgggtggatttggttctcgaaggcgatagctgcgtgactatcatgtctaaggacaagcctaccatcgatgtgaagatgatgaatatggaggcggccaacctggcagaggtccgcagttattgctatttggctaccgtcagcgatctctccaccaaagctgcgtgcccgaccatgggggaagcccacaatgacaaacgtgctgacccagcttttgtgtgcagacaaggagtggtggacaggggctggggcaacggctgcggactatttggcaaaggaagcattgacacatgcgccaaatttgcctgctctaccaaggcaataggaagaaccatcttgaaagagaatatcaagtacgaagtggccatttttgtccatggaccaactactgtggagtcgcacggaaactactccacacaggttggagccactcaggcagggagattcagcatcactcctgcggcgccttcatacacactaaagcttggagaatatggagaggtgacagtggactgtgaaccacggtcagggattgacaccaatgcatactacgtgatgactgttggaacaaagacgttcttggtccatcgtgagtggttcatggacctcaacctcccttggagcagtgctggaagtactgtgtggaggaacagagagacgttaatggagtttgaggaaccacacgccacgaagcagtctgtgatagcattgggctcacaagagggagctctgcatcaagctttggctggagccattcctgtggaattttcaagcaacactgtcaagttgacgtcgggtcatttgaagtgtagagtgaagatggaaaaattgcagttgaagggaacaacctatggcgtctgttcaaaggctttcaagtttcttgggactcccgcagacacaggtcacggcactgtggtgttggaattgcagtacactggcacggatggaccttgcaaagttcctatctcgtcagtggcttcattgaacgacctaacgccagtgggcagattggtcactgtcaacccttttgtttcagtggccacggccaacgctaaggtcctgattgaattggaaccaccctttggagactcatacatagtggtgggcagaggagaacaacagatcaatcaccattggcacaagtctggaagcagcattggcaaagcctttacaaccaccctcaaaggagcgcagagactagccgctctaggagacacagcttgggactttggatcagttggaggggtgttcacctcagttgggaaggctgtccatcaagtgttcggaggagcattccgctcactgttcggaggcatgtcctggataacgcaaggattgctgggggctctcctgttgtggatgggcatcaatgctcgtgataggtccatagctctcacgtttctcgcagttggaggagttctgctcttcctctccgtgaacgtgcacgctgacactgggtgtgccataaacatcacccggcaagagctgagatgtggaagtggagtgttcatacacaatgatgtggaggcttggatggaccggtacaagtattaccctgaaacgccacaaggcctagccaagatcattcagaaagctcataaggaaggagtgtgcggtctacgatcagtttccagactggagcatcaaatgtgggaagcagtgaaggacgagctgaacactcctttgaaggagaatggtgtggaccttagtgtcgtggttgagaaacaggagggaatgtacaagtcagcacctaaacgcctcaccgccaccacggaaaaattggaaattggctggaaggcctggggaaagagtattttatttgcaccagaactcgccaacaacacctttgtggttgatggtccggagaccaaggaatgtccgactcagaatcgcgcttggaatagcttagaagtggaggattttggatttggtctcaccagcactcggatgttcctgaaggtcagagaaggcaacacaactgaatgtgactcgaagatcattggaacggctgtcaagaacaacttggcgatccacagtgacctgtcctattggattgaaagcaggctcaatgatacgtggaagcttgaaagggcagttctgggtgaagtcaaatcatgtacgtggcctgagacgcataccttgtggggcgatggaatccttgagagtgacttgataataccagtcacactggcgggaccacgaagcaatcacaatcggagacctgggtacaagacacaaaaccagggcccatgggacgaaggccgggtagagattgacttcgattactgcccaggaactacggtcaccctgagtgagagctgcggacaccgtggacctgccactcgcaccaccacagagagcggaaagttgataacagattggtgctgcaggagctgcaccttaccaccactgcgctaccaaactgacagcggctgttggtatggtatggagatcagaccacagagacatgatgaaaagaccctcgtgcagtcacaagtgaatgcttataatgctgatatgattgacccttttcagttgggccttctggtcgtgttcttggccacccaggaggtccttcgcaagaggtggacagccaagatcagcatgccagctatactgattgctctgctagtcctggtgtttgggggcattacttacactgatgtgttacgctatgtcatcttggtgggggcagctttcgcagaatctaattcgggaggagacgtggtacacttggcgctcatggcgaccttcaagatacaaccagtgtttatggtggcatcgtttctcaaagcgagatggaccaaccaggagaacattttgttgatgttggcggctgttttctttcaaatggcttatcacgatgcccgccaaattctgctctgggagatccctgatgtgttgaattcactggcggtagcttggatgatactgagagccataacattcacaacgacatcaaacgtggttgttccgctgctagccctgctaacacccgggctgagatgcttgaatctggatgtgtacaggatactgctgttgatggtcggaataggcagcttgatcagggagaagaggagtgcagctgcaaaaaagaaaggagcaagtctgctatgcttggctctagcctcaacaggacttttcaaccccatgatccttgctgctggactgattgcatgtgatcccaaccgtaaacgcggatggcccgcaactgaagtgatgacagctgtcggcctaatgtttgccatcgtcggagggctggcagagcttgacattgactccatggccattccaatgactatcgcggggctcatgtttgctgctttcgtgatttctgggaaatcaacagatatgtggattgagagaacggcggacatttcctgggaaagtgatgcagaaattacaggctcgagcgaaagagttgatgtgcggcttgatgatgatggaaacttccagctcatgaatgatccaggagcaccttggaagatatggatgctcagaatggtctgtctcgcgattagtgcgtacaccccctgggcaatcttgccctcagtagttggattttggataactctccaatacacaaagagaggaggcgtgttgtgggacactccctcaccaaaggagtacaaaaagggggacaccaccaccggcgtttacaggatcatgactcgtgggctgctcggcagttatcaagcaggagcgggcgtgatggttgaaggtgttttccacaccctttggcatacaacaaaaggagccgctttgatgagcggagagggccgcctggacccatactggggcagtgtcaaggaggatcgactttgttacggaggaccctggaaattgcagcacaagtggaacgggcaggatgaggtgcagatgattgtggtggaacctggcaagaacgttaagaacgtccagacgaaaccaggggtgttcaaaacacctgaaggagaaatcggggccgtgactttggacttccccactggaacatcaggctcaccaatagtggacaaaaacggtgatgtgattgggctttatggcaatggagtcataatgcccaacggctcatacataagcgcgatagtgcagggtgaaaggatggatgagccaatcccagccggattcgaacctgagatgctgaggaaaaaacagatcactgtactggatctccatcccggcgccggtaaaacaaggaggattctgccacagatcatcaaagaggccataaacagaagactgagaacagccgtgctagcaccaaccagggttgtggctgctgagatggctgaagcactgagaggactgcccatccggtaccagacatccgcagtgcccagagaacataatggaaatgagattgttgatgtcatgtgtcatgctaccctcacccacaggctgatgtctcctcacagggtgccgaactacaacctgttcgtgatggatgaggctcatttcaccgacccagctagcattgcagcaagaggttacatttccacaaaggtcgagctaggggaggcggcggcaatattcatgacagccaccccaccaggcacttcagatccattcccagagtccaattcaccaatttccgacttacagactgagatcccggatcgagcttggaactctggatacgaatggatcacagaatacaccgggaagacggtttggtttgtgcctagtgtcaagatggggaatgagattgccctttgcctacaacgtgctggaaagaaagtagtccaattgaacagaaagtcgtacgagacggagtacccaaaatgtaagaacgatgattgggactttgttatcacaacagacatatctgaaatgggggctaacttcaaggcgagcagggtgattgacagccggaagagtgtgaaaccaaccatcataacagaaggagaagggagagtgatcctgggagaaccatctgcagtgacagcagctagtgccgcccagagacgtggacgtatcggtagaaatccgtcgcaagttggtgatgagtactgttatggggggcacacgaatgaagacgactcgaacttcgcccattggactgaggcacgaatcatgctggacaacatcaacatgccaaacggactgatcgctcaattctaccaaccagagcgtgagaaggtatataccatggatggggaataccggctcagaggagaagagagaaaaaactttctggaactgttgaggactgcagatctgccagtttggctggcttacaaggttgcagcggctggagtgtcataccacgaccggaggtggtgctttgatggtcctaggacaaacacaattttagaagacaacaacgaagtggaagtcatcacgaagcttggtgaaaggaagattctgaggccgcgctggattgacgccagggtgtactcggatcaccaggcactaaaggcgttcaaggacttcgcctcgggaaaacgttctcagatagggctcattgaggttctgggaaagatgcctgagcacttcatggggaagacatgggaagcacttgacaccatgtacgttgtggccactgcagagaaaggaggaagagctcacagaatggccctggaggaactgccagatgctcttcagacaattgccttgattgccttattgagtgtgatgaccatgggagtattcttcctcctcatgcagcggaagggcattggaaagataggtttgggaggcgctgtcttgggagtagcgacctttttctgttggatggctgaagttccaggaacgaagatcgccggaatgttgctgctctcccttctcttgatgattgtgctaattcctgagccagagaagcaacgttcgcagacagacaaccagctagccgtgttcctgatttgtgtcatgacccttgtgagcgcagtggcagccaacgagatgggttggctagataagaccaagagtgacataagcagtttgtttgggcaaagaattgaggtcaaggagaatttcagcatgggagagtttcttctggacttgaggccggcaacagcctggtcactgtacgctgtgacaacagcggtcctcactccactgctaaagcatttgatcacgtcagattacatcaacacctcattgacctcaataaacgttcaggcaagtgcactattcacactcgcgcgaggcttccccttcgtcgatgttggagtgtcggctctcctgctagcagccggatgctggggacaagtcaccctcaccgttacggtaacagcggcaacactccttttttgccactatgcctacatggttcccggttggcaagctgaggcaatgcgctcagcccagcggcggacagcggccggaatcatgaagaacgctgtagtggatggcatcgtggccacggacgtcccagaattagagcgcaccacacccatcatgcagaagaaagttggacagatcatgctgatcttggtgtctctagctgcagtagtagtgaacccgtctgtgaagacagtacgagaagccggaattttgatcacggccgcagcggtgacgctttgggagaatggagcaagctctgtttggaacgcaacaactgccatcggactctgccacatcatgcgtgggggttggttgtcatgtctatccataacatggacactcataaagaacatggaaaaaccagggctaaaaagaggtggggcaaaaggacgcaccttgggagaggtttggaaagaaagactcaaccagatgacaaaagaagagttcactaggtaccgcaaagaggccatcatcgaagtcgatcgctcagcggcaaaacacgccaggaaagaaggcaatgtcactggagggcatccagtctctaggggcacagcaaaactgagatggctggtcgaacggaggtttctcgaaccggtcggaaaagtgattgaccttggatgtggaagaggcggttggtgttactatatggcaacccaaaaaagagtccaagaagtcagagggtacacaaagggcggtcccggacatgaagagccccaactagtgcaaagttatggatggaacattgtcaccatgaagagtggagtggatgtgttctacagaccttctgagtgttgtgacaccctcctttgtgacatcggagagtcctcgtcaagtgctgaggttgaagagcataggacgattcgggtccttgaaatggttgaggactggctgcaccgagggccaagggaattttgcgtgaaggtgctctgcccctacatgccgaaagtcatagagaagatggagctgctccaacgccggtatggggggggactggtcagaaacccactctcacggaattccacgcacgagatgtattgggtgagtcgagcttcaggcaatgtggtacattcagtgaatatgaccagccaggtgctcctaggaagaatggaaaaaaggacctggaagggaccccaatacgaggaagatgtaaacttgggaagtggaaccagggcggtgggaaaacccctgctcaactcagacaccagtaaaatcaagaacaggattgaacgactcaggcgtgagtacagttcgacgtggcaccacgatgagaaccacccatatagaacctggaactaccacggcagttatgatgtgaagcccacaggctccgccagttcgctggtcaatggagtggtcaggctcctctcaaaaccatgggacaccatcacgaatgttaccaccatggccatgactgacactactcccttcgggcagcagcgagtgttcaaagagaaggtggacacgaaagctcctgaaccgccagaaggagtgaagtacgtgctcaacgagaccaccaactggttgtgggcgtttttggccagagaaaaacgtcccagaatgtgctctcgagaggaattcataagaaaggtcaacagcaatgcagctttgggtgccatgtttgaagagcagaatcaatggaggagcgccagagaagcagttgaagatccaaaattttgggagatggtggatgaggagcgcgaggcacatctgcggggggaatgtcacacttgcatttacaacatgatgggaaagagagagaaaaaacccggagagttcggaaaggccaagggaagcagagccatttggttcatgtggctcggagctcgctttctggagttcgaggctctgggttttctcaatgaagaccactggcttggaagaaagaactcaggaggaggtgtcgagggcttgggcctccaaaaactgggttacatcctgcgtgaagttggcacccggcctgggggcaagatctatgctgatgacacagctggctgggacacccgcatcacgagagctgacttggaaaatgaagctaaggtgcttgagctgcttgatggggaacatcggcgtcttgccagggccatcattgagctcacctatcgtcacaaagttgtgaaagtgatgcgcccggctgctgatggaagaaccgtcatggatgttatctccagagaagatcagagggggagtggacaagttgtcacctacgccctaaacactttcaccaacctggccgtccagctggtgaggatgatggaaggggaaggagtgattggcccagatgatgtggagaaactcacaaaagggaaaggacccaaagtcaggacctggctgtttgagaatggggaagaaagactcagccgcatggctgtcagtggagatgactgtgtggtaaagcccctggacgatcgctttgccacctcgctccacttcctcaatgctatgtcaaaggttcgcaaagacatccaagagtggaaaccgtcaactggatggtatgattggcagcaggttccattttgctcaaaccatttcactgaattgatcatgaaagatggaagaacactggtggttccatgccgaggacaggatgaattggtaggcagagctcgcatatctccaggggccggatggaacgtccgcgacactgcttgtctggctaagtcttatgcccagatgtggctgcttctgtacttccacagaagagacctgcggctcatggccaacgccatttgctccgctgtccctgtgaattgggtccctaccggaagaaccacgtggtccatccatgcaggaggagagtggatgacaacagaggacatgttggaggtctggaaccgtgtttggatagaggagaatgaatggatggaagacaaaaccccagtggagaaatggagtgacgtcccatattcaggaaaacgagaggacatctggtgtggcagcctgattggcacaagagcccgagccacgtgggcagaaaacatccaggtggctatcaaccaagtcagagcaatcatcggagatgagaagtatgtggattacatgagttcactaaagagatatgaagacacaactttggttgaggacacagtactgtagatatttaatcaattgtaaatagacaatataagtatgcataaaagtgtagttttatagtagtatttagtggtgttagtgtaaatagttaagaaaattttgaggagaaagtcaggccgggaagttcccgccaccggaagttgagtagacggtgctgcctgcgactcaaccccaggaggactgggtgaacaaagccgcgaagtgatccatgtaagccctcagaaccgtctcggaaggaggaccccacatgttgtaacttcaaagcccaatgtcagaccacgctacggcgtgctactctgcggagagtgcagtctgcgatagtgccccaggaggactgggttaacaaaggcaaaccaacgccccacgcggccctagccccggtaatggtgttaaccagggcgaaaggactagaggttagaggagaccccgcggtttaaagtgcacggcccagcctggctgaagctgtaggtcaggggaaggactagaggttagtggagaccccgtgccacaaaacaccacaacaaaacagcatattgacacctgggatagactaggagatcttctgctctgcacaaccagccacacggcacag

SEQ ID NO: 6 - DNA, 1542 bp, Escherichia coli 16S Ribosomal RNA
  misc_feature (896)..(896): n is a, c, g, or t
aaattgaagagtttgatcatggctcagattgaacgctggcggcaggcctaacacatgcaagtcgaacggtaacaggaakcagcttgctgatttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaaaggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtckaccgcctggggagtacggncgcaaggttaaaactcaaatgaattgacgggggcccgcacaagcggtggagcatgtggtttaattcgatgcaacgcgaagaaccttacctggtcttgacatccacagaactttccagagatggattggtgccttcgggaactgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgayctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 7 - DNA, 2905 bp, Escherichia coli 23 S Ribosomal RNA
ggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaagcgacttgctcgtggagctgaaatcagtcgaagataccagctggctgcaactgtttattaaaaacacagcactgtgcaaacacgaaagtggacgtatacggtgtgacgcctgcccggtgccggaaggttaattgatggggttagcggtaacgcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttggcccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatgcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcgcagcgatgcgttgagctaaccggtactaatgaaccgtgaggcttaacctt

SEQ ID NO: 8 - DNA, 1798 bp, Yeast (S. cerevisiae)
tatctggttgatcctgccagtagtcatatgcttgtctcaaagattaagccatgcatgtctaagtataagcaatttatacagtgaaactgcgaatggctcattaaatcagttatcgtttatttgatagttcctttactacatggtataaccgtggtaattctagagctaatacatgcttaaaatctcgaccctttggaagagatgtatttattagataaaaaatcaatgtcttcggactctttgatgattcataataacttttcgaatcgcatggccttgtgctggcgatggttcattcaaatttctgccctatcaactttcgatggtaggatagtggcctaccatggtttcaacgggtaacggggaataagggttcgattccggagagggagcctgagaaacggctaccacatccaaggaaggcagcaggcgcgcaaattacccaatcctaattcagggaggtagtgacaataaataacgatacagggcccattcgggtcttgtaattggaatgagtacaatgtaaataccttaacgaggaacaattggagggcaagtctggtgccagcagccgcggtaattccagctccaatagcgtatattaaagttgttgcagttaaaaagctcgtagttgaactttgggcccggttggccggtccgattttttcgtgtactggatttccaacggggcctttccttctggctaaccttgagtccttgtggctcttggcgaaccaggacttttactttgaaaaaattagagtgttcaaagcaggcgtattgctcgaatatattagcatggaataatagaataggacgtttggttctattttgttggtttctaggaccatcgtaatgattaatagggacggtcgggggcatcggtattcaattgtcgaggtgaaattcttggatttattgaagactaactactgcgaaagcatttgccaaggacgttttcattaatcaagaacgaaagttaggggatcgaagatgatctggtaccgtcgtagtcttaaccataaactatgccgactagatcgggtggtgtttttttaatgacccactcggtaccttacgagaaatcaaagtctttgggttctggggggagtatggtcgcaaggctgaaacttaaaggaattgacggaagggcaccactaggagtggagcctgcggctaatttgactcaacacggggaaactcaccaggtccagacacaataaggattgacagattgagagctctttcttgattttgtgggtggtggtgcatggccgtttctcagttggtggagtgatttgtctgcttaattgcgataacgaacgagaccttaacctactaaatagtggtgctagcatttgctggttatccacttcttagagggactatcggtttcaagccgatggaagtttgaggcaataacaggtctgtgatgcccttagaacgttctgggccgcacgcgcgctacactgacggagccagcgagtctaaccttggccgagaggtcttggtaatcttgtgaaactccgtcgtgctggggatagagcattgtaattattgctcttcaacgaggaattcctagtaagcgcaagtcatcagcttgcgttgattacgtccctgccctttgtacacaccgcccgtcgctagtaccgattgaatggcttagtgaggcctcaggatctgcttagagaagggggcaactccatctcagagcggagaatttggacaaacttggtcatttagaggaactaaaagtcgtaacaaggtttccgtaggtgaacctgcggaaggatcatta

SEQ ID NO: 9 - DNA, 3911 bp, Yeast 25S Ribosomal RNA
aattccgtgatgggcctttaggttttaccaactgcggctaatctttttttatactgagcgtattggaacgttatcgataagaagagagcgtctaggcgaacaatgttcttaaagtttgacctcaaatcaggtaggagtacccgctgaacttaagcatatcaataagcggaggaaaagaaaccaaccggattgccttagtaacggcgagtgaagcggcaaaagctcaaatttgaaatctggtaccttcggtgcccgagttgtaatttggagagggcaactttggggccgttccttgtctatgttccttggaacaggacgtcatagagggtgagcatcccgtgtggcgaggagtgcggttctttgtaaagtgccttcgaagagtcgagttgtttgggaatgcagctctaagtgggtggtaaattccatctaaagctaaatattggcgagagaccgatagcgaacaagtacagtgatggaaagatgaaaagaactttgaaaagagagtgaaaaagtacgtgaaattgttgaaagggaagggcatttgatcagacatggtgttttgtgccctctgctccttgtgggtaggggaatctcgcatttcactgggccagcatcagttttggtggcaggataaatccataggaatgtagcttgcctcggtaagtattatagcctgtgggaatactgccagctgggactgaggactgcgacgtaagtcaaggatgctggcataatggttatatgccgcccgtcttgaaacacggaccaaggagtctaacgtctatgcgagtgtttgggtgtaaaacccatacgcgtaatgaaagtgaacgtaggttggggcctcgcaagaggtgcacaatcgaccgatcctgatgtcttcggatggatttgagtaagagcatagctgttgggacccgaaagatggtgaactatgcctgaatagggtgaagccagaggaaactctggtggaggctcgtagcggttctgacgtgcaaatcgatcgtcgaatttgggtataggggcgaaagactaatcgaaccatctagtagctggttcctgccgaagtttccctcaggatagcagaagctcgtatcagttttatgaggtaaagcgaatgattagaggttccggggtcgaaatgaccttgacctattctcaaactttaaatatgtaagaagtccttgttacttaattgaacgtggacatttgaatgaagagcttttagtgggccatttttggtaagcagaactggcgatgcgggatgaaccgaacgtagagttaaggtgccggaatacacgctcatcagacaccacaaaaggtgttagttcatctagacagccggacggtggccatggaagtcggaatccgctaaggagtgtgtaacaactcaccggccgaatgaactagccctgaaaatggatggcgctcaagcgtgttacctatactctaccgtcagggttgatatgatgccctgacgagtaggcaggcgtggaggtcagtgacgaagcctagaccgtaaggtcgggtcgaacggcctctagtgcagatcttggtggtagtagcaaatattcaaatgagaactttgaagactgaagtggggaaaggttccacgtcaacagcagttggacgtgggttagtcgatcctaagagatggggaagctccgtttcaaaggcctgattttatgcaggccaccatcgaaagggaatccggtaagattccggaacttggatatggattcttcacggtaacgtaactgaatgtggagacgtcggcgcgagccctgggaggagttatcttttcttcttaacagcttatcaccccggaattggtttatccggagatggggtcttatggctggaagaggccagcacctttgctggctccggtgcgcttgtgacggcccgtgaaaatccacaggaaggaatagttttcatgctaggtcgtactgataaccgcagcaggtctccaaggtgaacagcctctagttgatagaataatgtagataagggaagtcggcaaaatagatccgtaacttcgggataaggattggctctaagggtcgggtagtgagggccttggtcagacgcagcgggcgtgcttgtggactgcttggtggggcttgctctgctaggcggactacttgcgtgccttgttgtagacggccttggtaggtctcttgtagaccgtcgcttgctacaattaacagatcaacttagaactggtacggacaaggggaatctgactgtctaattaaaacatagcattgcgatggtcagaaagtgatgttgacgcaatgtgatttctgcccagtgctctgaatgtcaaagtgaagaaattcaaccaagcgcgagtaaacggcgggagtaactatgactctcttaaggtagccaaatgcctcgtcatctaattagtgacgcgcatgaatggattaacgagattcccactgtccctatctactatctagcgaaaccacagccaagggaacgggcttggcagaatcagcggggaaagaagaccctgttgagcttgactctagtttgacattgtgaagagacatagagggtgtagaataagtgggagcttcggcgccagtgaaataccactacctttatagtttctttacttattcaatgaagcggagctggaattcattttccacgttctagcattcaaggtcccattcggggctgatccgggttgaagacattgtcaggtggggagtttggctggggcggcacatctgttaaacgataacgcagatgtcctaaggggggctcatggagaacagaaatctccagtagaacaaaagggtaaagccccttagtttgatttcagtgtgaatacaaaccattgaaagtgtggcctatcgatcctttagtccctcggaatttgaggctagaggtgccagaaaagttaccacagggataactggcttgtggcagtcaagcgttcatagcgacattgctttttgattcttcgatgtcggctcttcctatcataccgaagcagaattcggtaagcgttggattgttcacccactaatagggaacatgagctgggtttagaccgtcgtgagacaggttagttttaccctactgatgaatgttaccagcaatagtaattgaacttagtacgagaggaacagttcattcggataattggtttttgcggctgtctgatcaggcattgccgcgaagcaccatccgctggattatggctgaacgcctctaagtcagaatccatgctagaacgcggtgatttctttgctccacacaatatagatggatacgaataaggcgtccttgtggcgtcgctgaaccatagcaggctagcaacggtgcacttggcggaaaggccttgggtgcttgctggcgaattgcaatgtcattttgcgtggggataaatcatttgtatacgacttagatgtacaacggggtattgtaagcggtagagtagccttgttgttacgatctgctgagattaagcctttgttgtctgatttgttttttatttctttctaagtgggtactggcaggagccggggcctagtttagagagaagtagactcaacaagtctctataaattttatttgtcttaagaattctatgatccgggtaaaaacatgtattgtatatatctattataatatacgatgaggatgatagtgtgtaagagtgtaccatttactaatgtatgtaagttactatttactatttggtctttttattttttattttttttttttttttcgttgcaaagatgggttgaaagagaagggctttcacaaagcttcccgagcgtgaaaggatttgcccggacagtttgcttcatggagcagttttttccgcaccatcagagcggcaaacatgagtgcttgtataagtttagagaattgagaaaagctcattt

SEQ ID NO: 10 - DNA, 16569 bp, Human mitochondrial DNA
gatcacaggtctatcaccctattaaccactcacgggagctctccatgcatttggtattttcgtctggggggtatgcacgcgatagcattgcgagacgctggagccggagcaccctatgtcgcagtatctgtctttgattcctgcctcatcctattatttatcgcacctacgttcaatattacaggcgaacatacttactaaagtgtgttaattaattaatgcttgtaggacataataataacaattgaatgtctgcacagccactttccacacagacatcataacaaaaaatttccaccaaaccccccctcccccgcttctggccacagcacttaaacacatctctgccaaaccccaaaaacaaagaaccctaacaccagcctaaccagatttcaaattttatcttttggcggtatgcacttttaacagtcaccccccaactaacacattattttcccctcccactcccatactactaatctcatcaatacaacccccgcccatcctacccagcacacacacaccgctgctaaccccataccccgaaccaaccaaaccccaaagacaccccccacagtttatgtagcttacctcctcaaagcaatacactgaaaatgtttagacgggctcacatcaccccataaacaaataggtttggtcctagcctttctattagctcttagtaagattacacatgcaagcatccccgttccagtgagttcaccctctaaatcaccacgatcaaaagggacaagcatcaagcacgcagcaatgcagctcaaaacgcttagcctagccacacccccacgggaaacagcagtgattaacctttagcaataaacgaaagtttaactaagctatactaaccccagggttggtcaatttcgtgccagccaccgcggtcacacgattaacccaagtcaatagaagccggcgtaaagagtgttttagatcaccccctccccaataaagctaaaactcacctgagttgtaaaaaactccagttgacacaaaatagactacgaaagtggctttaacatatctgaacacacaatagctaagacccaaactgggattagataccccactatgcttagccctaaacctcaacagttaaatcaacaaaactgctcgccagaacactacgagccacagcttaaaactcaaaggacctggcggtgcttcatatccctctagaggagcctgttctgtaatcgataaaccccgatcaacctcaccacctcttgctcagcctatataccgccatcttcagcaaaccctgatgaaggctacaaagtaagcgcaagtacccacgtaaagacgttaggtcaaggtgtagcccatgaggtggcaagaaatgggctacattttctaccccagaaaactacgatagcccttatgaaacttaagggtcgaaggtggatttagcagtaaactaagagtagagtgcttagttgaacagggccctgaagcgcgtacacaccgcccgtcaccctcctcaagtatacttcaaaggacatttaactaaaacccctacgcatttatatagaggagacaagtcgtaacatggtaagtgtactggaaagtgcacttggacgaaccagagtgtagcttaacacaaagcacccaacttacacttaggagatttcaacttaacttgaccgctctgagctaaacctagccccaaacccactccaccttactaccagacaaccttagccaaaccatttacccaaataaagtataggcgatagaaattgaaacctggcgcaatagatatagtaccgcaagggaaagatgaaaaattataaccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagattaaaacactgaactgacaattaacagcccaatatctacaatcaaccaacaagtcattattaccctcactgtcaacccaacacaggcatgctcataaggaaaggttaaaaaaagtaaaaggaactcggcaaatcttaccccgcctgtttaccaaaaacatcacctctagcatcaccagtattagaggcaccgcctgcccagtgacacatgtttaacggccgcggtaccctaaccgtgcaaaggtagcataatcacttgttccttaaatagggacctgtatgaatggctccacgagggttcagctgtctcttacttttaaccagtgaaattgacctgcccgtgaagaggcgggcataacacagcaagacgagaagaccctatggagctttaatttattaatgcaaacagtacctaacaaacccacaggtcctaaactaccaaacctgcattaaaaatttcggttggggcgacctcggagcagaacccaacctccgagcagtacatgctaagacttcaccagtcaaagcgaactactatactcaattgatccaataacttgaccaacggaacaagttaccctagggataacagcgcaatcctattctagagtccatatcaacaatagggtttacgacctcgatgttggatcaggacatcccgatggtgcagccgctattaaaggttcgtttgttcaacgattaaagtcctacgtgatctgagttcagaccggagtaatccaggtcggtttctatctaccttcaaattcctccctgtacgaaaggacaagagaaataaggcctacttcacaaagcgccttcccccgtaaatgatatcatctcaacttagtattatacccacacccacccaagaacagggtttgttaagatggcagagcccggtaatcgcataaaacttaaaactttacagtcagaggttcaattcctcttcttaacaacatacccatggccaacctcctactcctcattgtacccattctaatcgcaatggcattcctaatgcttaccgaacgaaaaattctaggctatatacaactacgcaaaggccccaacgttgtaggcccctacgggctactacaacccttcgctgacgccataaaactcttcaccaaagagcccctaaaacccgccacatctaccatcaccctctacatcaccgccccgaccttagctctcaccatcgctcttctactatgaacccccctccccatacccaaccccctggtcaacctcaacctaggcctcctatttattctagccacctctagcctagccgtttactcaatcctctgatcagggtgagcatcaaactcaaactacgccctgatcggcgcactgcgagcagtagcccaaacaatctcatatgaagtcaccctagccatcattctactatcaacattactaataagtggctcctttaacctctccacccttatcacaacacaagaacacctctgattactcctgccatcatgacccttggccataatatgatttatctccacactagcagagaccaaccgaacccccttcgaccttgccgaaggggagtccgaactagtctcaggcttcaacatcgaatacgccgcaggccccttcgccctattcttcatagccgaatacacaaacattattataataaacaccctcaccactacaatcttcctaggaacaacatatgacgcactctcccctgaactctacacaacatattttgtcaccaagaccctacttctaacctccctgttcttatgaattcgaacagcatacccccgattccgctacgaccaactcatacacctcctatgaaaaaacttcctaccactcaccctagcattacttatatgatatgtctccatacccattacaatctccagcattccccctcaaacctaagaaatatgtctgataaaagagttactttgatagagtaaataataggagcttaaacccccttatttctaggactatgagaatcgaacccatccctgagaatccaaaattctccgtgccacctatcacaccccatcctaaagtaaggtcagctaaataagctatcgggcccataccccgaaaatgttggttatacccttcccgtactaattaatcccctggcccaacccgtcatctactctaccatctttgcaggcacactcatcacagcgctaagctcgcactgattttttacctgagtaggcctagaaataaacatgctagcttttattccagttctaaccaaaaaaataaaccctcgttccacagaagctgccatcaagtatttcctcacgcaagcaaccgcatccataatccttctaatagctatcctcttcaacaatatactctccggacaatgaaccataaccaatactaccaatcaatactcatcattaataatcataatagctatagcaataaaactaggaatagccccctttcacttctgagtcccagaggttacccaaggcacccctctgacatccggcctgcttcttctcacatgacaaaaactagcccccatctcaatcatataccaaatctctccctcactaaacgtaagccttctcctcactctctcaatcttatccatcatagcaggcagttgaggtggattaaaccagacccagctacgcaaaatcttagcatactcctcaattacccacataggatgaataatagcagttctaccgtacaaccctaacataaccattcttaatttaactatttatattatcctaactactaccgcattcctactactcaacttaaactccagcaccacgaccctactactatctcgcacctgaaacaagctaacatgactaacacccttaattccatccaccctcctctccctaggaggcctgcccccgctaaccggctttttgcccaaatgggccattatcgaagaattcacaaaaaacaatagcctcatcatccccaccatcatagccaccatcaccctccttaacctctacttctacctacgcctaatctactccacctcaatcacactactccccatatctaacaacgtaaaaataaaatgacagtttgaacatacaaaacccaccccattcctccccacactcatcgcccttaccacgctactcctacctatctccccttttatactaataatcttatagaaatttaggttaaatacagaccaagagccttcaaagccctcagtaagttgcaatacttaatttctgtaacagctaaggactgcaaaaccccactctgcatcaactgaacgcaaatcagccactttaattaagctaagcccttactagaccaatgggacttaaacccacaaacacttagttaacagctaagcaccctaatcaactggcttcaatctacttctcccgccgccgggaaaaaaggcgggagaagccccggcaggtttgaagctgcttcttcgaatttgcaattcaatatgaaaatcacctcggagctggtaaaaagaggcctaacccctgtctttagatttacagtccaatgcttcactcagccattttacctcacccccactgatgttcgccgaccgttgactattctctacaaaccacaaagacattggaacactatacctattattcggcgcatgagctggagtcctaggcacagctctaagcctccttattcgagccgagctgggccagccaggcaaccttctaggtaacgaccacatctacaacgttatcgtcacagcccatgcatttgtaataatcttcttcatagtaatacccatcataatcggaggctttggcaactgactagttcccctaataatcggtgcccccgatatggcgtttccccgcataaacaacataagcttctgactcttacctccctctctcctactcctgctcgcatctgctatagtggaggccggagcaggaacaggttgaacagtctaccctcccttagcagggaactactcccaccctggagcctccgtagacctaaccatcttctccttacacctagcaggtgtctcctctatcttaggggccatcaatttcatcacaacaattatcaatataaaaccccctgccataacccaataccaaacgcccctcttcgtctgatccgtcctaatcacagcagtcctacttctcctatctctcccagtcctagctgctggcatcactatactactaacagaccgcaacctcaacaccaccttcttcgaccccgccggaggaggagaccccattctataccaacacctattctgatttttcggtcaccctgaagtttatattcttatcctaccaggcttcggaataatctcccatattgtaacttactactccggaaaaaaagaaccatttggatacataggtatggtctgagctatgatatcaattggcttcctagggtttatcgtgtgagcacaccatatatttacagtaggaatagacgtagacacacgagcatatttcacctccgctaccataatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagcccacttccactatgtcctatcaataggagctgtatttgccatcataggaggcttcattcactgatttcccctattctcaggctacaccctagaccaaacctacgccaaaatccatttcactatcatattcatcggcgtaaatctaactttcttcccacaacactttctcggcctatccggaatgccccgacgttactcggactaccccgatgcatacaccacatgaaacatcctatcatctgtaggctcattcatttctctaacagcagtaatattaataattttcatgatttgagaagccttcgcttcgaagcgaaaagtcctaatagtagaagaaccctccataaacctggagtgactatatggatgccccccaccctaccacacattcgaagaacccgtatacataaaatctagacaaaaaaggaaggaatcgaaccccccaaagctggtttcaagccaaccccatggcctccatgactttttcaaaaaggtattagaaaaaccatttcataactttgtcaaagttaaattataggctaaatcctatatatcttaatggcacatgcagcgcaagtaggtctacaagacgctacttcccctatcatagaagagcttatcacctttcatgatcacgccctcataatcattttccttatctgcttcctagtcctgtatgcccttttcctaacactcacaacaaaactaactaatactaacatctcagacgctcaggaaatagaaaccgtctgaactatcctgcccgccatcatcctagtcctcatcgccctcccatccctacgcatcctttacataacagacgaggtcaacgatccctcccttaccatcaaatcaattggccaccaatggtactgaacctacgagtacaccgactacggcggactaatcttcaactcctacatacttcccccattattcctagaaccaggcgacctgcgactccttgacgttgacaatcgagtagtactcccgattgaagcccccattcgtataataattacatcacaagacgtcttgcactcatgagctgtccccacattaggcttaaaaacagatgcaattcccggacgtctaaaccaaaccactttcaccgctacacgaccgggggtatactacggtcaatgctctgaaatctgtggagcaaaccacagtttcatgcccatcgtcctagaattaattcccctaaaaatctttgaaatagggcccgtatttaccctatagcaccccctctaccccctctagagcccactgtaaagctaacttagcattaaccttttaagttaaagattaagagaaccaacacctctttacagtgaaatgccccaactaaatactaccgtatggcccaccataattacccccatactccttacactattcctcatcacccaactaaaaatattaaacacaaactaccacctacctccctcaccaaagcccataaaaataaaaaattataacaaaccctgagaaccaaaatgaacgaaaatctgttcgcttcattcattgcccccacaatcctaggcctacccgccgcagtactgatcattctatttccccctctattgatccccacctccaaatatctcatcaacaaccgactaatcaccacccaacaatgactaatcaaactaacctcaaaacaaatgataaccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcacagtgattataggctttcgctctaagattaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaagcgccaccctagcaatatcaaccattaaccttccctctacacttatcatcttcacaattctaattctactgactatcctagaaatcgctgtcgccttaatccaagcctacgttttcacacttctagtaagcctctacctgcacgacaacacataatgacccaccaatcacatgcctatcatatagtaaaacccagcccatgacccctaacaggggccctctcagccctcctaatgacctccggcctagccatgtgatttcacttccactccataacgctcctcatactaggcctactaaccaacacactaaccatataccaatgatggcgcgatgtaacacgagaaagcacataccaaggccaccacacaccacctgtccaaaaaggccttcgatacgggataatcctatttattacctcagaagtttttttcttcgcaggatttttctgagccttttaccactccagcctagcccctaccccccaattaggagggcactggcccccaacaggcatcaccccgctaaatcccctagaagtcccactcctaaacacatccgtattactcgcatcaggagtatcaatcacctgagctcaccatagtctaatagaaaacaaccgaaaccaaataattcaagcactgcttattacaattttactgggtctctattttaccctcctacaagcctcagagtacttcgagtctcccttcaccatttccgacggcatctacggctcaacattttttgtagccacaggcttccacggacttcacgtcattattggctcaactttcctcactatctgcttcatccgccaactaatatttcactttacatccaaacatcactttggcttcgaagccgccgcctgatactggcattttgtagatgtggtttgactatttctgtatgtctccatctattgatgagggtcttactcttttagtataaatagtaccgttaacttccaattaactagttttgacaacattcaaaaaagagtaataaacttcgccttaattttaataatcaacaccctcctagccttactactaataattattacattttgactaccacaactcaacggctacatagaaaaatccaccccttacgagtgcggcttcgaccctatatcccccgcccgcgtccctttctccataaaattcttcttagtagctattaccttcttattatttgatctagaaattgccctccttttacccctaccatgagccctacaaacaactaacctgccactaatagttatgtcatccctcttattaatcatcatcctagccctaagtctggcctatgagtgactacaaaaaggattagactgaaccgaattggtatatagtttaaacaaaacgaatgatttcgactcattaaattatgataatcatatttaccaaatgcccctcatttacataaatattatactagcatttaccatctcacttctaggaatactagtatatcgctcacacctcatatcctccctactatgcctagaaggaataatactatcgctgttcattatagctactctcataaccctcaacacccactccctcttagccaatattgtgcctattgccatactagtctttgccgcctgcgaagcagcggtgggcctagccctactagtctcaatctccaacacatatggcctagactacgtacataacctaaacctactccaatgctaaaactaatcgtcccaacaattatattactaccactgacatgactttccaaaaaacacataatttgaatcaacacaaccacccacagcctaattattagcatcatccctctactattttttaaccaaatcaacaacaacctatttagctgttccccaaccttttcctccgaccccctaacaacccccctcctaatactaactacctgactcctacccctcacaatcatggcaagccaacgccacttatccagtgaaccactatcacgaaaaaaactctacctctctatactaatctccctacaaatctccttaattataacattcacagccacagaactaatcatattttatatcttcttcgaaaccacacttatccccaccttggctatcatcacccgatgaggcaaccagccagaacgcctgaacgcaggcacatacttcctattctacaccctagtaggctcccttcccctactcatcgcactaatttacactcacaacaccctaggctcactaaacattctactactcactctcactgcccaagaactatcaaactcctgagccaataacttaatatgactagcttacacaatagcttttatagtaaagatacctctttacggactccacttatgactccctaaagcccatgtcgaagcccccatcgctgggtcaatagtacttgccgcagtactcttaaaactaggcggctatggtataatacgcctcacactcattctcaaccccctgacaaaacacatagcctaccccttccttgtactatccctatgaggcataattataacaagctccatctgcctacgacaaacagacctaaaatcgctcattgcatactcttcaatcagccacatagccctcgtagtaacagccattctcatccaaaccccctgaagcttcaccggcgcagtcattctcataatcgcccacgggcttacatcctcattactattctgcctagcaaactcaaactacgaacgcactcacagtcgcatcataatcctctctcaaggacttcaaactctactcccactaatagctttttgatgacttctagcaagcctcgctaacctcgccttaccccccactattaacctactgggagaactctctgtgctagtaaccacgttctcctgatcaaatatcactctcctacttacaggactcaacatactagtcacagccctatactccctctacatatttaccacaacacaatggggctcactcacccaccacattaacaacataaaaccctcattcacacgagaaaacaccctcatgttcatacacctatcccccattctcctcctatccctcaaccccgacatcattaccgggttttcctcttgtaaatatagtttaaccaaaacatcagattgtgaatctgacaacagaggcttacgaccccttatttaccgagaaagctcacaagaactgctaactcatgcccccatgtctaacaacatggctttctcaacttttaaaggataacagctatccattggtcttaggccccaaaaattttggtgcaactccaaataaaagtaataaccatgcacactactataaccaccctaaccctgacttccctaattccccccatccttaccaccctcgttaaccctaacaaaaaaaactcatacccccattatgtaaaatccattgtcgcatccacctttattatcagtctcttccccacaacaatattcatgtgcctagaccaagaagttattatctcgaactgacactgagccacaacccaaacaacccagctctccctaagcttcaaactagactacttctccataatattcatccctgtagcattgttcgttacatggtccatcatagaattctcactgtgatatataaactcagacccaaacattaatcagttcttcaaatatctactcatcttcctaattaccatactaatcttagttaccgctaacaacctattccaactgttcatcggctgagagggcgtaggaattatatccttcttgctcatcagttgatgatacgcccgagcagatgccaacacagcagccattcaagcaatcctatacaaccgtatcggcgatatcggtttcatcctcgccttagcatgatttatcctacactccaactcatgagacccacaacaaatagcccttctaaacgctaatccaagcctcaccccactactaggcctcctcctagcagcagcaggcaaatcagcccaattaggtctccacccctgactcccctcagccatagaaggccccaccccagtctcagccctactccactcaagcactatagttgtagcaggaatcttcttactcatccgcttccaccccctagcagaaaatagcccactaatccaaactctaacactatgcttaggcgctatcaccactctgttcgcagcagtctgcgcccttacacaaaatgacatcaaaaaaatcgtagccttctccacttcaagtcaactaggactcataatagttacaatcggcatcaaccaaccacacctagcattcctgcacatctgtacccacgccttcttcaaagccatactatttatgtgctccgggtccatcatccacaaccttaacaatgaacaagatattcgaaaaataggaggactactcaaaaccatacctctcacttcaacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataacctattcccccgagcaatctcaattacaatatatacaccaacaaacaatgttcaaccagtaactactactaatcaacgcccataatcatacaaagcccccgcaccaataggatcctcccgaatcaaccctgacccctctccttcataaattattcagcttcctacactattaaagtttaccacaaccaccaccccatcatactctttcacccacagcaccaatcctacctccatcgctaaccccactaaaacactcaccaagacctcaacccctgacccccatgcctcaggatactcctcaatagccatcgctgtagtatatccaaagacaaccatcattccccctaaataaattaaaaaaactattaaacccatataacctcccccaaaattcagaataataacacacccgaccacaccgctaacaatcaatactaaacccccataaataggagaaggcttagaagaaaaccccacaaaccccattactaaacccacactcaacagaaacaaagcatacatcattattctcgcacggactacaaccacgaccaatgatatgaaaaaccatcgttgtatttcaactacaagaacaccaatgaccccaatacgcaaaattaaccccctaataaaattaattaaccactcattcatcgacctccccaccccatccaacatctccgcatgatgaaacttcggctcactccttggcgcctgcctgatcctccaaatcaccacaggactattcctagccatgcactactcaccagacgcctcaaccgccttttcatcaatcgcccacatcactcgagacgtaaattatggctgaatcatccgctaccttcacgccaatggcgcctcaatattctttatctgcctcttcctacacatcgggcgaggcctatattacggatcatttctctactcagaaacctgaaacatcggcattatcctcctgcttgcaactatagcaacagccttcataggctatgtcctcccgtgaggccaaatatcattctgaggggccacagtaattacaaacttactatccgccatcccatacattgggacagacctagttcaatgaatctgaggaggctactcagtagacagtcccaccctcacacgattctttacctttcacttcatcttgcccttcattattgcagccctagcaacactccacctcctattcttgcacgaaacgggatcaaacaaccccctaggaatcacctcccattccgataaaatcaccttccacccttactacacaatcaaagacgccctcggcttacttctcttccttctctccttaatgacattaacactattctcaccagacctcctaggcgacccagacaattataccctagccaaccccttaaacacccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcctgtccttgtagtataaactaatacaccagtcttgtaaaccggagatgaaaacctttttccaaggacaaatcagagaaaaagtctttaactccaccattagcacccaaagctaagattctaatttaaactattctctgttctttcatggggaagcagatttgggtaccacccaagtattgactcacccatcaacaaccgctatgtatttcgtacattactgccagccaccatgaatattgtacggtaccataaatacttgaccacctgtagtacataaaaacccaatccacatcaaaaccccctccccatgcttacaagcaagtacagcaatcaaccctcaactatcacacatcaactgcaactccaaagccacccctcacccactaggataccaacaaacctacccacccttaacagtacatagtacataaagccatttaccgtacatagcacattacagtcaaatcccttctcgtccccatggatgacccccctcagataggggtcccttgaccaccatcctccgtgaaatcaatatcccgcacaagagtgctactctcctcgctccgggcccataacacttgggggtagctaaagtgaactgtatccgacatctggttcctacttcagggtcataaagcctaaatagcccacacgttccccttaaataagacatcacgatg

SEQ ID NO: 11 - DNA, 17 bp, B19 Virus
tggtctgggatgaaggt

SEQ ID NO: 12 - DNA, 23 bp, B19 virus
ccattttaggcgggcaacccacc

SEQ ID NO: 13 - DNA, 21 bp, B19 virus
tggaagtgtagctgtgcctgg

SEQ ID NO: 14 - DNA, 23 bp, B19 virus
agaatcatttgtcggaagctcag

SEQ ID NO: 15 - DNA, 20 bp, B19 virus
acaagcctgggcaagttagc

SEQ ID NO: 16 - DNA, 21 bp, B19 virus
acaatgccagtggaaaggagg

SEQ ID NO: 17 - DNA, 23 bp, B19 virus
ctttaacacatgaagaccatgca

SEQ ID NO: 18 - DNA, 28 bp, B19 virus
cctctcaaaacactagaatatccttacg

SEQ ID NO: 19 - DNA, 27 bp, B19 virus
cagccataccaccactgggacacagat

SEQ ID NO: 20 - DNA, 23 bp, B19 virus
aatgccatttctcatggtcagac

SEQ ID NO: 21 - DNA, 20 bp, B19 virus
gcaaaacaacaccacaggca

SEQ ID NO: 22 - DNA, 22 bp, Hepatitis B virus
caacctccaatcactcaccaac

SEQ ID NO: 23 - DNA, 24 bp, Hepatitis B virus
cctccaatttgtcctggttatcgc

SEQ ID NO: 24 - DNA, 23 bp, Hepatitis B virus
gtgtctgcggcgttttatcatat

SEQ ID NO: 25 - DNA, 23 bp, Hepatitis B virus
tgtttggctttcagctatatgga

SEQ ID NO: 26 - DNA, 22 bp, Hepatitis B virus
aattgtgggtcttttgggcttt

SEQ ID NO: 27 - DNA, 18 bp, Hepatitis B virus
ttctccgtctgccgttcc

SEQ ID NO: 28 - DNA, 21 bp, Hepatitis B virus
accagcaccatgcaacttttt

SEQ ID NO: 29 - DNA, 25 bp, Hepatitis B virus
tttttcacctctgcctaatcatctc

SEQ ID NO: 30 - DNA, 16 bp, Hepatitis B virus
tcccactgttcaagcc

SEQ ID NO: 31 - DNA, 21 bp, Hepatitis B virus
acctcaccataccgcactcag

SEQ ID NO: 32 - DNA, 25 bp, Hepatitis B virus
gggaattgatgactctagctacctg

SEQ ID NO: 33 - DNA, 22 bp, Hepatitis B virus
atcctgaatggcaaactccttc

SEQ ID NO: 34 - DNA, 20 bp, Hepatitis B virus
gagaaaccacacgtagcgca

SEQ ID NO: 35 - DNA, 16 bp, Hepatitis B virus
cacccctccacacggc

SEQ ID NO: 36 - DNA, 27 bp, Hepatitis B virus
atctctccacctctaagagacagtcat

SEQ ID NO: 37 - DNA, 22 bp, Porcine parvovirus
cctcacaaaacggcaagtactg

SEQ ID NO: 38 - DNA, 36 bp, Porcine parvovirus
acctaagtccaagtgactgctactggttcatacagc

SEQ ID NO: 39 - DNA, 29 bp, Porcine parvovirus
gttaataatgcaatgcaaagtacctctaa

SEQ ID NO: 40 - DNA, 25 bp, Porcine parvovirus
aaagacaactgaaagagagcatgga

SEQ ID NO: 41 - DNA, 30 bp, Porcine parvovirus
tctcagctcagattctggcttcatgacaaa

SEQ ID NO: 42 - DNA, 22 bp, Porcine parvovirus
caattctatttcatgggccagc

SEQ ID NO: 43 - DNA, 17 bp, Porcine parvovirus
cgtggagcgagccaaca

SEQ ID NO: 44 - DNA, 28 bp, Porcine parvovirus
ctgcacttaactccaacaccgccagatt

SEQ ID NO: 45 - DNA, 24 bp, Porcine parvovirus
gcaatacggacaccaagtccaact

SEQ ID NO: 46 - DNA, 23 bp, Porcine parvovirus
gaggtaagaagatcgccgagaaa

SEQ ID NO: 47 - DNA, 28 bp, Porcine parvovirus
aacctcaccaccaaccaaaatatataat

SEQ ID NO: 48 - DNA, 29 bp, Porcine parvovirus
actactaactgaacctaccacagaaggag

SEQ ID NO: 49 - DNA, 26 bp, Porcine parvovirus
cttttaccttcagatccaataggagg

SEQ ID NO: 50 - DNA, 18 bp, Sindbis virus
gcgtgcggaccctgtact

SEQ ID NO: 51 - DNA, 25 bp, Sindbis virus
attggcttcgacaccacccagttca

SEQ ID NO: 52 - DNA, 20 bp, Sindbis virus
ttctcggctatggcaggttc

SEQ ID NO: 53 - DNA, 25 bp, Sindbis virus
gtttatttctccgtaggatcgacac

SEQ ID NO: 54 - DNA, 20 bp, Sindbis virus
aaaactgctgcaggtctcgg

SEQ ID NO: 55 - DNA, 22 bp, Sindbis virus
gaaatcgatattacaggggcca

SEQ ID NO: 56 - DNA, 21 bp, Sindbis virus
gcattaagtttttcggcatgg

SEQ ID NO: 57 - DNA, 18 bp, Sindbis virus
cgattggcatagccggtg

SEQ ID NO: 58 - DNA, 21 bp, West Nile virus
tcagcgatctctccaccaaag

SEQ ID NO: 59 - DNA, 22 bp, West Nile virus
tgcccgaccatgggggaagccc

SEQ ID NO: 60 - DNA, 21 bp, West Nile virus
caatgacaaacgtgctgaccc

SEQ ID NO: 61 - DNA, 20 bp, West Nile virus
gctagtcctggtgtttgggg

SEQ ID NO: 62 - DNA, 24 bp, Escherichia coli 16S Ribosomal RNA
agagtttgatcatggctcagattg

SEQ ID NO: 63 - DNA, 24 bp, Escherichia coli 16S Ribosomal RNA
ctggcggcaggcctaacacatgca

SEQ ID NO: 64 - DNA, 21 bp, Escherichia coli 16S Ribosomal RNA
aataccgcataacgtcgcaag

SEQ ID NO: 65 - DNA, 20 bp, Escherichia coli 16S Ribosomal RNA
gatgcaacgcgaagaacctt

SEQ ID NO: 66 - DNA, 23 bp, Escherichia coli 16S Ribosomal RNA
gactggggtgaagtcgtaacaag

SEQ ID NO: 67 - DNA, 24 bp, Escherichia coli 16S Ribosomal RNA
gtaacaaggtaaccgtaggggaac

SEQ ID NO: 68 - DNA, 19 bp, Escherichia coli 16S Ribosomal RNA
gcggttggatcacctcctt

SEQ ID NO: 69 - DNA, 22 bp, Escherichia coli 23S Ribosomal RNA
ccgatagtgaaccagtaccgtg

SEQ ID NO: 70 - DNA, 25 bp, Escherichia coli 23S Ribosomal RNA
atgttgaaaaattagcggatgactt

SEQ ID NO: 71 - DNA, 18 bp, Escherichia coli 23S Ribosomal RNA
gcactgtttcggcaaggg

SEQ ID NO: 72 - DNA, 18 bp, Escherichia coli 23S Ribosomal RNA
gccggaagaccaagggtt

SEQ ID NO: 73 - DNA, 24 bp, Escherichia coli 23S Ribosomal RNA
ggccgtaactataacggtcctaag

SEQ ID NO: 74 - DNA, 26 bp, Escherichia coli 23S Ribosomal RNA
gataagtgctgaaagcatctaagcac

SEQ ID NO: 75 - DNA, 25 bp, Yeast (S. cerevisiae)
ctgccagtagtcatatgcttgtctc

SEQ ID NO: 76 - DNA, 36 bp, Yeast (S. cerevisiae)
tacagtgaaactgcgaatggctcattaaatcagtta

SEQ ID NO: 77 - DNA, 27 bp, Yeast (S. cerevisiae)
taatacatgcttaaaatctcgaccctt

SEQ ID NO: 78 - DNA, 24 bp, Yeast (S. cerevisiae)
gtcttcggactctttgatgattca

SEQ ID NO: 79 - DNA, 17 bp, Yeast (S. cerevisiae)
gcagccgcggtaattcc

SEQ ID NO: 80 - DNA, 24 bp, Yeast (S. cerevisiae)
gctgaaacttaaaggaattgacgg

SEQ ID NO: 81 - DNA, 23 bp, Yeast (S. cerevisiae)
tggaagtttgaggcaataacagg

SEQ ID NO: 82 - DNA, 19 bp, Yeast (S. cerevisiae)
tgaacctgcggaaggatca

SEQ ID NO: 83 - DNA, 24 bp, Yeast 25S Ribosomal RNA
aagcatatcaataagcggaggaaa

SEQ ID NO: 84 - DNA, 20 bp, Yeast 25S Ribosomal RNA
ctctggtggaggctcgtagc

SEQ ID NO: 85 - DNA, 18 bp, Yeast 25S Ribosomal RNA
aatggatggcgctcaagc

SEQ ID NO: 86 - DNA, 25 bp, Yeast 25S Ribosomal RNA
tgaaaatccacaggaaggaatagtt

SEQ ID NO: 87 - DNA, 21 bp, Yeast 25S Ribosomal RNA
ctaagggtcgggtagtgaggg

SEQ ID NO: 88 - DNA, 20 bp, Yeast 25S Ribosomal RNA
agaaattcaaccaagcgcga

SEQ ID NO: 89 - DNA, 19 bp, Yeast 25S Ribosomal RNA
atgtcattttgcgtgggga

SEQ ID NO: 90 - DNA, 23 bp, Human mitochondrial DNA
gttcaccctctaaatcaccacga

SEQ ID NO: 91 - DNA, 23 bp, Human mitochondrial DNA
caagcacgcagcaatgcagctca

SEQ ID NO: 92 - DNA, 26 bp, Human mitochondrial DNA
ggaaacagcagtgattaacctttagc

SEQ ID NO: 93 - DNA, 28 bp, Human mitochondrial DNA
gactacgaaagtggctttaacatatctg

SEQ ID NO: 94 - DNA, 24 bp, Human mitochondrial DNA
tagagtgcttagttgaacagggcc

SEQ ID NO: 95 - DNA, 24 bp, Human mitochondrial DNA
taggcgatagaaattgaaacctgg

SEQ ID NO: 96 - DNA, 21 bp, Human mitochondrial DNA
tttgttaagatggcagagccc

SEQ ID NO: 97 - DNA, 20 bp, Human mitochondrial DNA
agaatcgaacccatccctga

SEQ ID NO: 98 - DNA, 19 bp, Human mitochondrial DNA
tttcaccgtaggtggcctg

SEQ ID NO: 99 - DNA, 20 bp, Human mitochondrial DNA
aatcgctgtcgccttaatcc